(19) United States
(12) Patent Application Publication
(10) Pub. No.: US 2019/0388679 A1
(43) Pub. Date: Dec. 26, 2019

(54) METHOD AND SYSTEM FOR ANALYZING INVASIVE BRAIN STIMULATIONS

(71) Applicants: Elminda Ltd., Herzliya (IL); Yissum Research Development Company of the Hebrew University of Jersusalem Ltd., Jerusalem (IL)

(72) Inventors: Amir B. GEVA, Tel-Aviv (IL); Hagai BERGMAN, Jerusalem (IL); Ziv PEREMEN, Natanya (IL); Dror HAOR, Kibbutz Hulda (IL); Daniel SAND, Ramat-HaSharon (IL); Yaki STERN, Moshav Sde Yaakov (IL); Boaz SADEH, Rehovot (IL)

(73) Assignees: Elminda Ltd., Herzliya (IL); Yissum Research Development Company of the Hebrew University of Jersusalem Ltd., Jerusalem (IL)

(21) Appl. No.: 16/490,907

(22) PCT Filed: Mar. 7, 2018
(86) PCT No.: PCT/IL2018/050267

§ 371 Date: Sep. 4, 2019

Related U.S. Application Data
(60) Provisional application No. 62/467,905, filed on Mar. 7, 2017.

(51) Int. Cl. A61N 1/05; A61B 5/04; A61B 5/0478; A61B 5/00; A61B 5/048

(52) U.S. Cl. CPC A61N 1/0534; A61B 5/04008; A61B 5/0484; A61B 5/4848; A61B 5/048; A61B 5/0478

(57) ABSTRACT

A method of analyzing performance of a brain stimulation tool is disclosed. The method comprises: obtaining encephalography data collected from a brain of a subject electrically stimulated by at least one of the electrode contacts of the brain stimulation tool; segmenting the data into a plurality of epochs, each corresponding to a single stimulation event generated by the brain stimulation tool; and applying a spatiotemporal analysis to the epochs so as to determine at least one of (i) location of the at least one electrode contact in the brain, and (ii) therapeutic effect of the at least one electrode contact.

### FIELD AND BACKGROUND OF THE INVENTION

The present invention, in some embodiments thereof, relates to neuroscience and, more particularly, but not exclusively, to a method and system for analyzing brain stimulations generated by a brain stimulation tool. The analysis can, in some embodiments of the present invention, be used for configuring the brain stimulation tool.

A wide variety of mental and physical processes are controlled or influenced by neural activity in particular regions of the brain. For example, various physical or cognitive functions are directed or affected by neural activity within the sensory or motor cortices. Across most individuals, particular areas of the brain appear to have distinct functions. In the majority of people, for example, the areas of the occipital lobes relate to vision; the regions of the left interior frontal lobes relate to language; portions of the cerebral cortex appear to be consistently involved with conscious awareness, memory, and intellect; and particular regions of the cerebral cortex as well as the basal ganglia, the thalamus and the motor cortex cooperatively interact to facilitate motor function control.

A movement disorder is a neurological disturbance that involves one or more muscles or muscle groups, and may include simple or complex movements and actions. Movement disorders include Parkinson's disease, Huntington's Chorea, progressive supranuclear palsy, Wilson's disease, Tourette's syndrome, epilepsy, and various chronic tremors, tics and dystonias. Different clinically observed movement disorders can be traced to the same or similar areas of the brain. Abnormalities of basal ganglia, for example, are postulated as a causative factor in diverse movement disorders. More specifically, deficiency of the neurotransmitter dopamine as the consequence of degenerative, vascular or inflammatory changes in the basal ganglia is postulated as the main reason for development of Parkinson's disease. Clinical symptoms of the Parkinson's disease, such as rhythmical muscular tremors, rigidity of movement, festination, droopy posture and masklike facies, are known to appear after 50-60% neuronal loss has occurred in the dopamine neurons of the substantia nigra compacta.

Tremors are characterized by abnormal, involuntary movements. An essential tremor is maximal when the body part afflicted (often an arm or hand) is being used, for example when attempts at writing or fine coordinated hand movements are made (postural tremor). A resting tremor is common in Parkinson's disease and in syndromes with Parkinsonian features. A resting tremor is maximal when the extremities are at rest. Often, when a patient attempts fine movement, such as reaching for a cup, the tremor subsides. Dystonias are involuntary movement disorders characterized by continued muscular contractions which can result in twisted contorted postures involving the body or limbs. Causes of dystonia include genetic biochemical abnormalities, degenerative disorders, psychiatric dysfunction, toxins, drugs and central trauma.

There are a wide variety of treatment modalities for neurological disease in general and movement disorders in particular. These include the use of medicaments (e.g., dopaminergic agonists or anticholinergic agents), tissue ablation (e.g., pallidotomy, thalamotomy, subthalamotomy, gammaknife, focused ultrasound and other radiofrequency lesioning procedures), and tissue transplantation (e.g., animal or human mesencephalic cells).

Another approach is by electrical stimulation of a predetermined neurological region. The use of electrical stimulation for treating neurological disease, including movement disorders, has been widely discussed in the literature. It has been recognized that electrical stimulation holds significant advantages over radiofrequency lesioning, inasmuch as radiofrequency lesioning can only destroy nervous system tissue. In many instances, the preferred effect is to stimulate to increase, decrease or block neuronal activity. Electrical stimulation permits such modulation of the target neural structures and, equally importantly, does not require the destruction of nervous tissue. It can also be adjusted to the changes in the disease conditions

A variety of brain-controlled disorders, including movement disorders, have been found treatable via electrical treatment with deep brain stimulation (DBS). Many disabling symptoms of the Parkinson's disease, including tremor, muscular rigidity, dyskinesia, bradykinesia are known to be effectively treated with a DBS electrode, when traditional medical treatment fails. The neurostimulation blocks the symptoms of the disease resulting in increased quality of life for the patient.

Generally DBS involves placement of a permanent DBS electrode having two or more (e.g., four) electrode contacts through burr holes drilled in the patient's skull, and then applying appropriate stimulation through the electrode contacts to the physiological targets. The various contacts of the electrode typically penetrate into different regions (e.g., at different depths). In a typical DBS electrode, the contacts are numbered according to their distance from the proximal end of the electrode. For example, in a four contact DBS electrode, the contacts are conventionally numbered from 0 to 3 (for the right side) or from 8 to 11 (for the left side), where contact Nos. 0 and 8 are the botomost contacts (farthest from the proximal end) and contact Nos. 3 and 11 are the topmost contacts (closests to the proximal end).

To date, DBS has been successfully used for treatment of movement disorders in the Vim nucleus, the globus pallidus internal segment (GPi), and the subthalamic nucleus (STN). DBS is particularly effective for relieving of tremors, rigidity, bradykinesia, and dyskinesia.

A typical DBS system comprises a programmable pulse generator, also referred to as a neurostimulator, operatively connected to the brain by one or more DBS electrodes with electrode contacts positioned for the desired stimulation. The electrode contacts are placed in the nervous tissue by a stereotaxic operation whereby each DBS electrode is placed in the desired target with an accuracy of a few millimeters. Typically, the process of implantation of a DBS electrode follows a step-wise progression of (i) initial estimation of target localization based on imaged anatomical landmarks; (ii) intra-operative micro-physiological mapping of key features associated with the intended target of interest; (iii) verification of the implantation site by assessment of the therapeutic window of stimulation; and (iv) implantation of the DBS electrode with the contacts located at the final desired target.

Several DBS systems have been developed over the years. To this end see, e.g., U.S. Pat. Nos. 5,515,848, 5,843,093, 6,560,472, 6,799,074, 6,011,996, 6,094,598, 6,760,626, 6,950,709 and 7,010,356; U.S. Patent Applications No. 20020022872, 20020198446, 20050015130, 20050165465, 20050246004, 2005055064, 20060069415, 20060041284, 20060089697; and International Patent Applications, Publication Nos. WO 1999/036122, WO 2002/011703 and WO 2006/034305.

### SUMMARY OF THE INVENTION

According to an aspect of some embodiments of the present invention there is provided a method of analyzing performance of a brain stimulation tool having a plurality of electrode contacts. The method comprises: obtaining encephalography data collected from a brain of a subject electrically stimulated by at least one of the electrode contacts; segmenting the data into a plurality of epochs, each corresponding to a single stimulation event generated by the brain stimulation tool; and applying a spatiotemporal analysis to the epochs so as to determine at least one of (i) location of the at least one electrode contact in the brain, and (ii) therapeutic effect of the at least one electrode contact.

According to some embodiments of the invention the single stimulation event is generated by a single pulse applied by a single electrode contact.

According to some embodiments of the invention the single stimulation event is generated by more than one electrode contacts, each applying a single pulse.

According to some embodiments of the invention the encephalography data comprise EEG data.

According to some embodiments of the invention the encephalography data comprise MEG data.

According to some embodiments of the invention the electrode contacts are electrode contacts of at least one DBS electrode.

According to some embodiments of the invention the segmenting comprises extracting stimulation pulses onsets from the data based on at least one of shapes and patterns of artifacts in the data.

According to some embodiments of the invention the brain of the subject is stimulated at a frequency of at most 20 Hz, wherein each epoch has a duration of at least 50 ms. According to some embodiments of the invention the brain of the subject is stimulated at a frequency of at most 10 Hz, wherein each epoch has a duration of at least 100 ms. According to some embodiments of the invention the brain of the subject is stimulated at a frequency of at most 5 Hz, wherein each epoch has a duration of at least 200 ms.

According to some embodiments of the invention the brain of the subject is stimulated by one electrode contact at a time. According to some embodiments of the invention the brain of the subject is stimulated by two electrode contacts at a time, simultaneously. According to some embodiments of the invention the brain of the subject is stimulated by three electrode contacts at a time, simultaneously.

According to some embodiments of the invention each stimulation event is characterized by a set of parameters, wherein all stimulation events are characterized by the same set of values for the parameters.

According to some embodiments of the invention the method comprises repeating the operations of obtaining, segmenting, and applying spatiotemporal analysis for a different set of values for the parameters.

According to some embodiments of the invention the parameters comprise at least one of: stimulation intensity, stimulation frequency, and stimulation directionality.

According to some embodiments of the invention the spatiotemporal analysis comprises: identifying activity-related features in the epochs; parceling the data according to the activity-related features to define a plurality of capsules, each representing a spatiotemporal activity region in the brain; and comparing capsules corresponding to different electrode contacts; wherein the determination of the location and/or therapeutic effect is based, at least in part, on the comparison.

According to some embodiments of the invention the comparison comprises calculating a similarity score among pairs of capsules.

According to some embodiments of the invention the method comprises clustering the capsules to provide at least one cluster of capsules, wherein the determination of the location and/or therapeutic effect is based, at least in part, on a size of the at least one cluster.

According to some embodiments of the invention the method comprises configuring a neurostimulator of the brain stimulation tool, based on the location and/or therapeutic effect.

According to some embodiments of the invention the method comprises applying a time-frequency analysis to the epochs to provide time-frequency patterns, wherein the determination of the location is based on the time-frequency patterns.

According to an aspect of some embodiments of the present invention there is provided a method of analyzing performance of a brain stimulation tool having a plurality of electrode contacts. The method comprises: obtaining encephalography data collected from a brain of a subject electrically stimulated by at least one of the electrode contacts; segmenting the data into a plurality of epochs, each corresponding to a stimulation event generated by a train of pulses transmitted by single electrode contact; and calculating power spectral density for averages of the epochs so as to determine location of the at least one electrode contact in the brain.

According to some embodiments of the invention the brain of the subject is stimulated intermittently at a frequency of at least 80 Hz. According to some embodiments of the invention the brain of the subject is stimulated intermittently at a frequency of at least 90 Hz. According to some embodiments of the invention the brain of the subject is stimulated intermittently at a frequency of at least 100 Hz. According to some embodiments of the invention the brain of the subject is stimulated intermittently at a frequency of at least 110 Hz. According to some embodiments of the invention the brain of the subject is stimulated intermittently at a frequency of at least 120 Hz. According to some embodiments of the invention the brain of the subject is stimulated intermittently at a frequency of at least 130 Hz.

According to some embodiments of the invention the method comprises determining distribution of the encephalography data over a scalp of the subject, separately for at least one encephalographic frequency band, wherein the determination of the location is also based on the distribution.

According to some embodiments of the invention the DBS elctrode contacts are implanted to apply at a location selected from the group consisting of the Vim nucleus, the globus pallidus internal segment (GPi), and the subthalamic nucleus (STN). According to some embodiments of the invention the DBS electrode contacts are implanted to treat at least one movement disorder selected from the group consisting of, tremors, rigidity, bradykinesia, dyskinesia, and/or at least one non-motor disorder selected from the group consisting of depression, obsessive-compulsive disorder, chronic pain, traumatic brain injury (tbi) and post-traumatic stress disorder.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIG. 1 shows deep brain stimulation evoked response in the subthalamic nucleus of a brain, as obtained in experiments performed according to some embodiments of the present invention;

FIG. 2 shows deep brain stimulation evoked response in the globus pallidus of a brain, as obtained in experiments performed according to some embodiments of the present invention;

FIG. 3 shows scalp topography analysis of deep brain stimulation evoked response in the globus pallidus of a brain, as obtained in experiments performed according to some embodiments of the present invention;

FIG. 4 is a schematic illustration describing a Spatiotemporal parcellation process used in experiments performed according to some embodiments of the present invention according to some embodiments of the invention;

FIG. 5 shows clustering of results obtained according to some embodiments of the present invention by the Spatiotemporal parcellation process;

FIG. 6 is a schematic dimensional illustration of Spatiotemporal parcellation similarity measure, used in experiments performed according to some embodiments of the present invention;

FIGS. 7A-D show two-dimensional similarity measures for contact Nos. 0 and 1 (FIG. 7A), contact Nos. 0 and 2 (FIG. 7B), contact Nos. 1 and 2 (FIG. 7C), and contact Nos. 0 and 3 (FIG. 7D), obtained in experiments performed according to some embodiments of the present invention.

FIG. 7E shows capsules that were obtained at t=240 ms and that correspond to the similarity measures shows in FIGS. 7A-D.

FIGS. 8A-H show maps of Event Related Spectral Dynamics obtained according to some embodiments of the present invention

FIGS. 9A-I show PSD analysis of one electrode performed on a subject according to some embodiments of the present invention;

FIGS. 10A-I show scalp topography maps of the PSD analyses, as performed according to some embodiments of the present invention;

FIG. 11 shows the potential in μV as a function of the time for the selected ROI, as obtained according to some embodiments of the present invention at stimulation frequency of 5 Hz;

FIG. 12 shows the potential in μV as a function of the time for the selected ROI, as obtained according to some embodiments of the present invention at stimulation frequency of 2 Hz;

FIG. 13 shows a one-way analysis of variance of the area under a curve as calculated according to some embodiments of the present invention;

FIG. 14 is a flowchart diagram of a method suitable for analyzing neurophysiological data, according to various exemplary embodiments of the present invention;

FIG. 15 is a schematic illustration showing a representative example of a Brain Network Activity (BNA) pattern which can be extracted from neurophysiological data, according to some embodiments of the present invention;

FIG. 16A is a flowchart diagram describing a procedure for identifying activity-related features for a group of subjects, according to some embodiments of the present invention;

FIG. 16B is schematic illustration of a procedure for determining relations between brain activity features, according to some embodiments of the present invention;

FIGS. 16C-E are abstract illustrations of a BNA patterns constructed according to some embodiments of the present invention using the procedure illustrated in FIG. 16B;

FIG. 17 is a flowchart diagram illustrating a method suitable for constructing a database from neurophysiological data recorded from a group of subjects, according to some embodiments of the present invention;

FIG. 18 is a flowchart diagram illustrating another method suitable for analyzing neurophysiological data recorded from a subject, according to some embodiments of the present invention;

FIG. 19 which is a flowchart diagram of a method suitable for analyzing performance of an invasive stimulation tool, according to various exemplary embodiments of the present invention; and

FIG. 20 is a schematic illustration of a system suitable for analyzing performance of an invasive brain stimulation tool having a plurality of electrode contacts, and optionally also for treating a subject by the invasive brain stimulation tool, according to some embodiments of the present invention.

### DESCRIPTION OF SPECIFIC EMBODIMENTS OF THE INVENTION

The present invention, in some embodiments thereof, relates to neuroscience and, more particularly, but not exclusively, to a method and system for analyzing brain stimulations generated by a brain stimulation tool. The analysis can, in some embodiments of the present invention, be used for configuring the brain stimulation tool.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

Embodiments of the present invention are directed to a technique for estimating potential distribution on a surface using electrical potential measured on another surface, and the electrical property distribution and geometry of a volume between the two surfaces. In any of the embodiments described herein, the surface is preferably the cortical surface of a brain of a subject, e.g., a mammalian subject, preferably a human subject. Optionally, but not necessarily, the estimated potential distribution is thereafter used for assessing a change in a condition of the brain and/or the effect of a particular treatment applied to the subject.

It is to be understood that, unless otherwise defined, the operations described hereinbelow can be executed either contemporaneously or sequentially in many combinations or orders of execution. Specifically, the ordering of the flowchart diagrams is not to be considered as limiting. For example, two or more operations, appearing in the following description or in the flowchart diagrams in a particular order, can be executed in a different order (e.g., a reverse order) or substantially contemporaneously. Additionally, several operations described below are optional and may not be executed.

At least part of the operations can be can be implemented by a data processing system, e.g., a dedicated circuitry or a general purpose computer, configured for receiving the data and executing the operations described below. At least part of the operations can be can be implemented by a cloud-computing facility at a remote location.

Computer programs implementing the method of the present embodiments can commonly be distributed to users on a distribution medium such as, but not limited to, a floppy disk, a CD-ROM, a flash memory device and a portable hard drive. From the distribution medium, the computer programs can be copied to a hard disk or a similar intermediate storage medium. The computer programs can be run by loading the computer instructions either from their distribution medium or their intermediate storage medium into the execution memory of the computer, configuring the computer to act in accordance with the method of this invention. All these operations are well-known to those skilled in the art of computer systems.

The method of the present embodiments can be embodied in many forms. For example, it can be embodied in on a tangible medium such as a computer for performing the method operations. It can be embodied on a computer readable medium, comprising computer readable instructions for carrying out the method operations. In can also be embodied in electronic device having digital computer capabilities arranged to run the computer program on the tangible medium or execute the instruction on a computer readable medium.

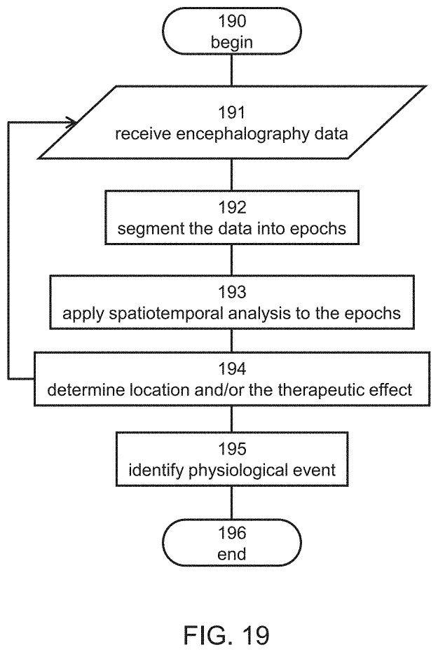

Reference is now made to FIG. 19 which is a flowchart diagram of a method suitable for analyzing performance of an invasive stimulation tool, according to various exemplary embodiments of the present invention. The invasive stimulation tool can be, for example, an invasive brain stimulation tool, such as, but not limited to, a Deep Brain Stimulation (DBS) tool, an invasive spinal cord stimulation tool, an invasive vagal nerve stimulation tool, and an invasive peripheral nerve stimulation tool. The invasive stimulation tool preferably has one or more electrodes, with two or three or four or more contacts per electrode.

The method begins at 190 and optionally and preferably continues to 191 at which encephalography (EG) data collected from a brain of a subject electrically stimulated by one or more of the electrode contacts are obtained. In any of the embodiments described herein, the EG data can include electroencephalogram data (EEG data), magnetoencephalogram data (MEG data), both EEG data and MEG data, a combination (e.g., an average, a weighted average,) of EEG data and MEG data normalized to allow such combination or a selective local substitution of either EEG data or MEG data based on some criterion or set of criteria, following for example, a statistical analysis of the EEG data or MEG data at each measuring location.

The EG data are recorded from a scalp surface of the subject's head, and the brain of the subject can stimulated during and/or before the collection of the EG data from the brain. The stimulation can be at any frequency. Preferably, but not necessarily the frequency is less than the frequency that is typically applied during an ongoing treatment. For example, when the tool is a DBS tool, a typical ongoing treatment includes stimulation at a frequency of above 100 Hz. In this case, the subject is stimulated during and/or before the collection of the EG data from the brain at a frequency which is less than 100 Hz or less than 80 Hz or less than 60 Hz. In some embodiments of the present invention the stimulation is at a frequency of at least 5 Hz or at least 10 Hz or at least 20 Hz or at least 30 Hz. Also contemplated, are embodiments in which the EG data are collected from the brain during an ongoing invasive treatment. Thus, the present embodiments also contemplate the subject is stimulated during and/or before the collection of the EG data from the brain at a frequency which is at least 80 Hz or at least 90 Hz or at least 100 Hz or at least 110 Hz or at least 120 Hz or at least 130 Hz.

The brain of the subject can be stimulated by one electrode contact at a time. In other embodiments, the brain of subject is stimulated by two electrode contacts at a time, simultaneously. In additional embodiments, the brain of the subject is stimulated by three electrode contacts at a time, simultaneously. A stimulation event can be applied by the respective electrode contacts as a continuous wave stimulation, or as a pulse, or as a train of pulses.

Each stimulation event is characterized by a set of parameters, including, without limitation, frequency, voltage, directionality, pulse repetition rate, pulse width, and the electrode contact or contacts that is or are used for applying the stimulation. Preferably, the EG data are collected from the brain during a plurality of stimulation events, wherein all stimulation events are characterized by the same set of values for the parameters.

The EG data can include a plurality of waveforms, which are typically time domain waveforms, where each waveform corresponds to a different EG channel and describes electrical potentials measured at a different location over the scalp. One or more of the waveforms, preferably all the waveforms can also be decomposed into a plurality of partial waveforms each corresponding to different frequency range within the waveform. The EG data can be either received from an external source (for example, a data storage system storing the EG data, optionally and preferably in a digitized form, on a suitable storage medium), or it can be measured by the method using an EG system having EG electrodes connected to the scalp, and an EG measuring device that receives electrical signals from the electrodes and converts the signals to EG data, optionally and preferably digitized EG data.

The method optionally and preferably continues to 192 at which the data are segmented into a plurality of epochs, each corresponding to a single stimulation event generated by the brain stimulation tool, more preferably by one contact of one electrode. Preferably, one or more of the single stimulation event is generated by a single pulse applied by a single electrode contact. Also contemplated, are embodiments in which one or more of the stimulation events is generated by more than one electrode contacts, where each electrode contact applies a single pulse. The segmentation can include receiving a timing schedule of the stimulation and aligning the data and the timing schedule so that the epochs correspond to stimulation events. The segmentation can optionally and preferably comprise extracting stimulation pulses onsets from the EG data based on shapes and/or patterns of artifacts in the data. Combination of these techniques is also contemplated.

The method continues to 193 at which a spatiotemporal analysis is applied to epochs so as to determine 194 the location of one or more of the electrode contacts in brain, and/or the therapeutic effect of one or more of the electrode contacts in brain. The spatiotemporal analysis can be applied so as to determine whether the contact that corresponds to an epoch or set of epochs is outside or inside a particular region in the organ to which the stimulation is applied, or in which of a set of regions in the organ the contact is located. For example, when the stimulation is applied to the brain, spatiotemporal analysis can be applied so as to determine whether or not the contact is inside one of the STN and GP, and/or to determine in which of the STN and GP the electrode contact is located, or in which of the segments (internal, external) of the GP the electrode contact is located, or whether or not the electrode contact is located inside or outside the GPi.

Generally, the spatiotemporal analysis can construct a spatiotemporal object, such as, but not limited to, a brain network activity (BNA) pattern with a plurality of nodes, each representing an activity feature in the data encompassed by the epoch, or a capsule representing a spatiotemporal activity region in the brain. The BNA pattern or capsule can then be used for determining the location and/or the therapeutic effect of the electrode contact. Representative examples of techniques for constructing BNA patterns and capsules are described in greater detail in Annex 1 and Annex 2, below.

Typically, the method constructs a spatiotemporal object for each of the electrode contacts and then compares the spatiotemporal objects of different electrode contacts to provide similarity scores, as described in Annex 1 and 2 below. When the similarity score of the spatiotemporal objects of two different electrode contacts is above a predetermined threshold, the method can determine that both contacts are at the same location and have a therapeutic effect.

For one or more of the constructed spatiotemporal objects, the method can compare the constructed spatiotemporal object, with a reference spatiotemporal object, such as, but not limited to, a reference spatiotemporal object that is an entry of a library of reference spatiotemporal objects stored on a computer-readable medium. The reference spatiotemporal object can be annotated according to the expected location for which such reference spatiotemporal object is typical. In these embodiments, the method can estimate the location of the contact that corresponds to the respective epoch or set of epochs based on a similarity score between the spatiotemporal object and the annotated reference spatiotemporal object, wherein a similarity score above a predetermined threshold indicates that the location of the contact is at the expected location that annotates the reference spatiotemporal object.

The reference spatiotemporal object can alternatively or additionally be annotated according to the expected therapeutic effect for which such reference spatiotemporal object is typical. In these embodiments, the method can estimate the therapeutic effect of the contact that corresponds to the respective epoch or set of epochs based on a similarity score between the spatiotemporal object and the annotated reference spatiotemporal object, wherein a similarity score above a predetermined threshold indicates that the therapeutic effect of the contact is at expected therapeutic effect that annotates the reference spatiotemporal object.

The method can also compare a spatiotemporal object constructed from an epoch corresponding to a stimulation event, with a spatiotemporal object constructed from an epoch corresponding to a rest period in which the stimulation was off. In this case, the method optionally and preferably provides a dissimilarity score. When the dissimilarity score of two such spatiotemporal objects is above a predetermined threshold, the method can determine that the respective contact has a therapeutic effect.

In some embodiments of the present invention the spatiotemporal objects are clustered to provide a cluster of spatiotemporal objects (e.g., a cluster of BNA patterns or a cluster of capsules). In these embodiments the determination of location and/or therapeutic effect is based, at least in part, on a size of the cluster. For example, when a cluster includes a large number of spatiotemporal objects of the same electrode contact, the method can determine that the respective electrode contact has a therapeutic effect. When a cluster includes a large number of spatiotemporal objects of two electrode contacts, the method can determine that the respective electrode contacts are at the same location in the brain. In some embodiments of the present invention the spatiotemporal analysis includes constructing multidimensional vectors with cross contact similarity scores. In these embodiments, the clustering can be of the multidimensional vectors instead of the spatiotemporal objects.

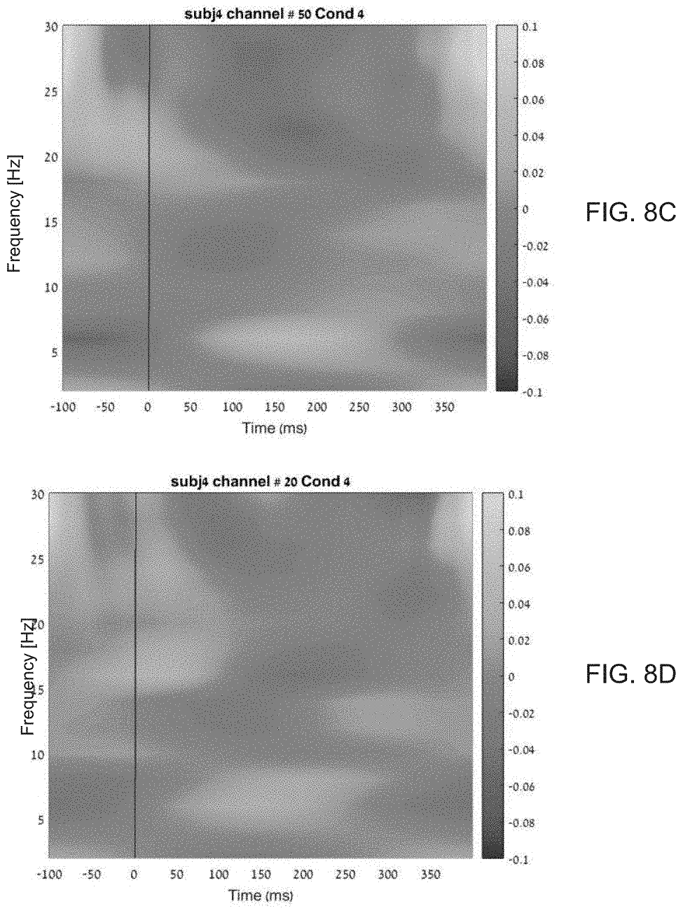

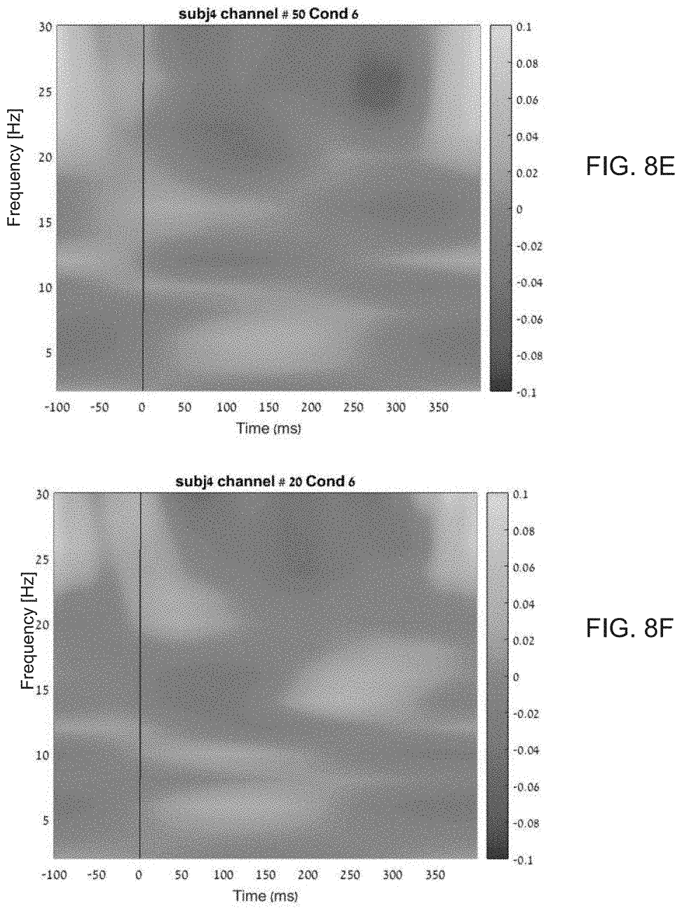

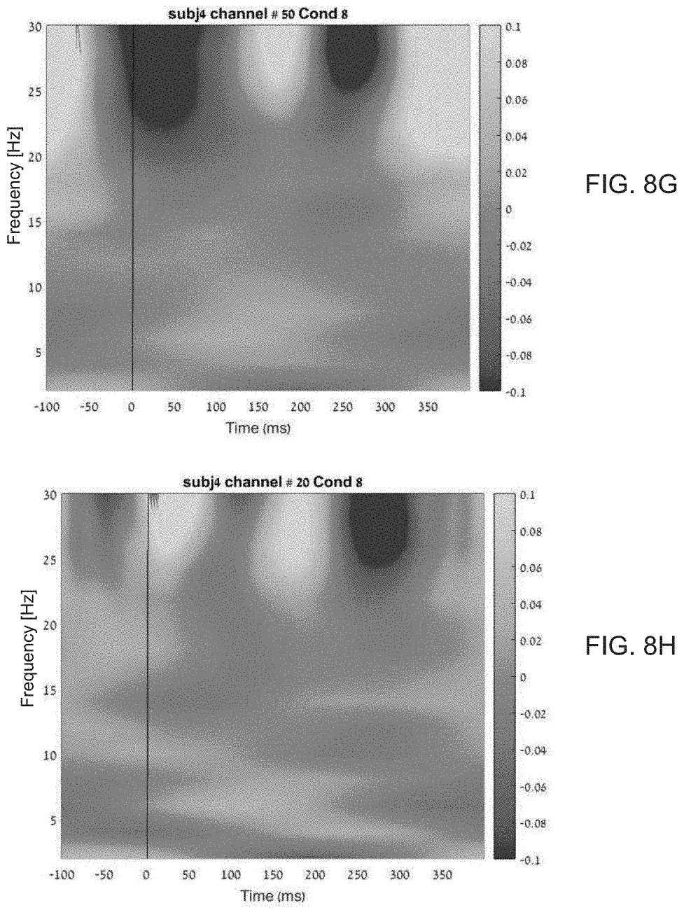

The present embodiments also contemplate the use of other objects for the analysis. For example, a time-frequency analysis can be applied to the epochs to provide time-frequency patterns. The time-frequency patterns can also be used for the determination of the location and/or the therapeutic effect. Use of time-frequency patterns for the determination of the location and/or the therapeutic effect is demonstrated in FIGS. 8A-H of the Examples section that follows.

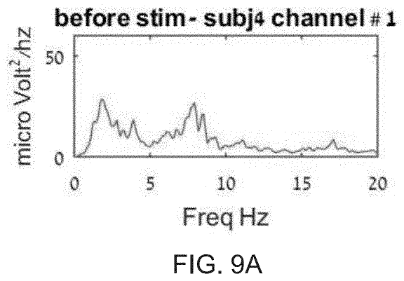

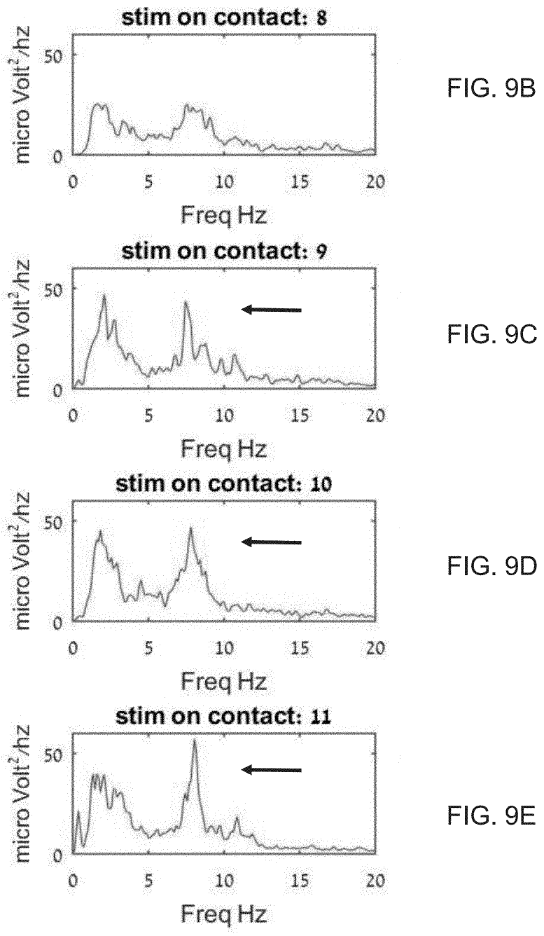

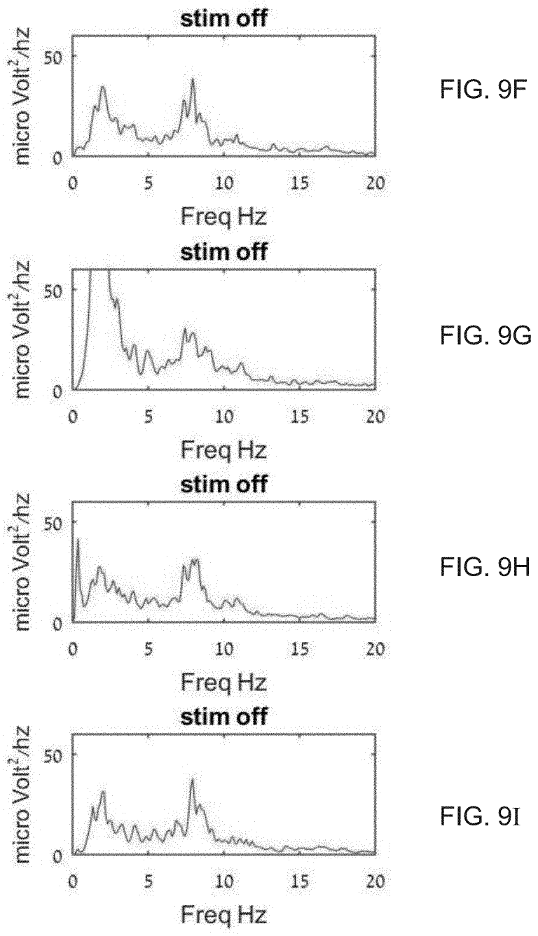

When the stimulation includes a train of pulses transmitted by single electrode contact, a power spectral density can be optionally and preferably calculated for averages of epochs, where each epoch corresponds to a stimulation event that is a train of pulses. The power spectral density can also be used for the determination of the location and/or the therapeutic effect. Use of power spectral density for the determination of the location and/or the therapeutic effect is demonstrated in FIGS. 9A-I of the Examples section that follows.

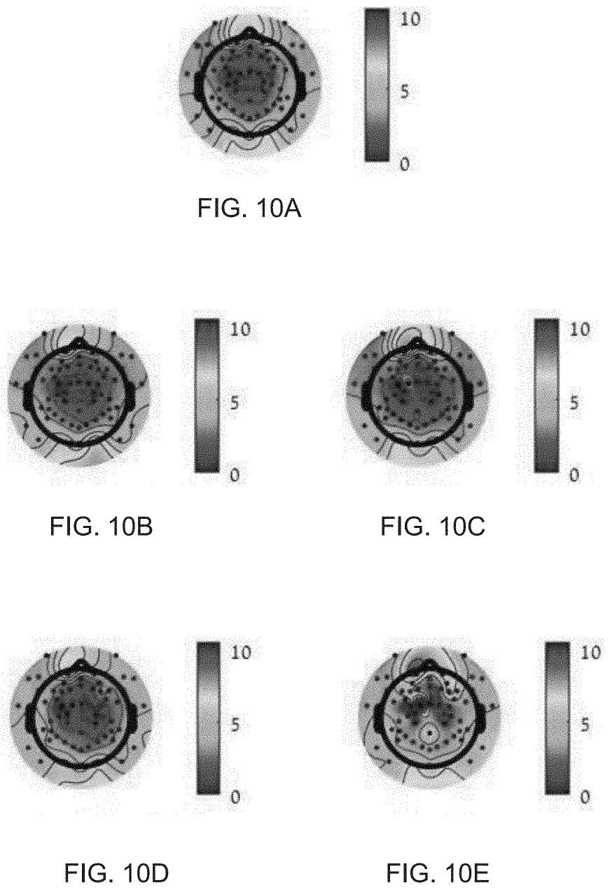

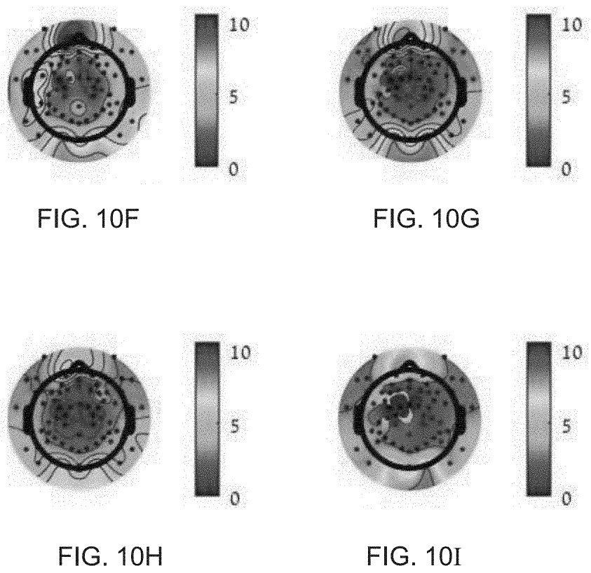

Also contemplated, are embodiments in which the distribution of EG data over the scalp of subject is determined separately for each encephalographic frequency band. The distribution can be used for the determination of the location and/or the therapeutic effect. Use of such distribution is demonstrated in FIGS. 10A-I of the Examples section that follows.

From 194 the method can loop back to 191 to receive EG data corresponding to stimulation events at a different set of parameters and to execute at least some of operations 192, 193 and 194 for the new set of parameters.

In some embodiments of the present invention the method continues to 195 at which the spatiotemporal analysis and/or time-frequency analysis is used to identify a physiological event, such as, but not limited to, increased tremor, and increased twitching. This can be done by comparing the spatiotemporal object to a baseline and detecting an abrupt change of the spatiotemporal object relative to the baseline.

The method ends at 196.

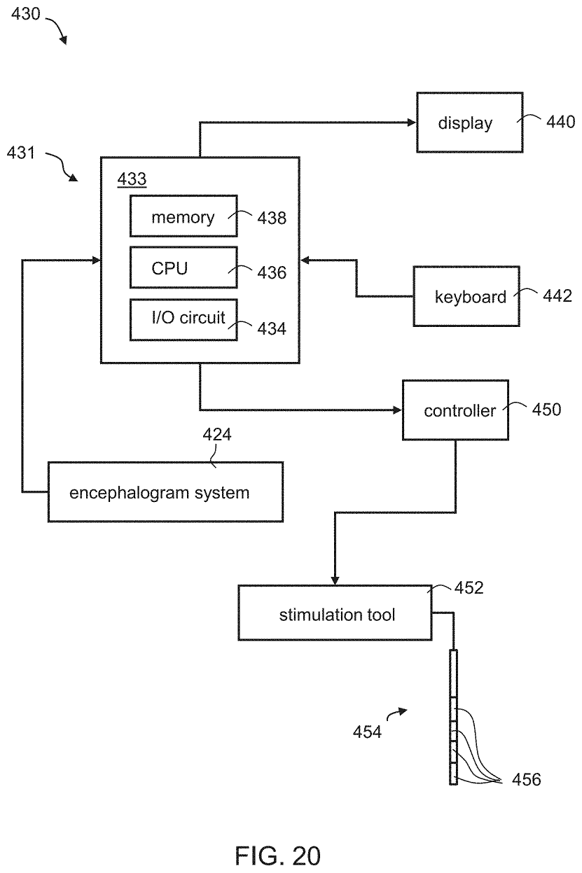

FIG. 20 is a schematic illustration of a system 430 suitable for analyzing performance of an invasive brain stimulation tool having a plurality of electrode contacts, and optionally also for treating a subject by the invasive brain stimulation tool, according to some embodiments of the present invention. System 430 typically comprises a data processing system 431, which can comprise a computer 433, which typically comprises an input/output (I/O) circuit 434, a data processor, such as a central processing unit (CPU) 436 (e.g., a microprocessor), and a memory 446 which typically includes both volatile memory and non-volatile memory. I/O circuit 434 is used to communicate information in appropriately structured form to and from other CPU 436 and other devices or networks external to system 430. CPU 436 is in communication with I/O circuit 434 and memory 438. These elements are those typically found in most general purpose computers and are known per se.

A display device 440 is shown in communication with computer 433, typically via I/O circuit 434. Computer 433 issues to display device 440 graphical and/or textual output images generated by CPU 436. A keyboard 442 is also shown in communication with computer 433, typically I/O circuit 434.

It will be appreciated by one of ordinary skill in the art that system 431 can be part of a larger system. For example, system 431 can also be in communication with a network, such as connected to a local area network (LAN), the Internet or a cloud computing resource of a cloud computing facility.

Data processing system 431 is preferably configured for analyzing performance of an invasive brain stimulation tool, for example, by executing method 190.

In some optional embodiments of the present invention, system 430 comprises or is in communication with an EG system 424 (e.g., an EEG system, an MEG system or a combined EEG-MEG system) configured for sensing and/or recording the EG data and feeding data processor 433 with the data.

In some optional embodiments of the present invention system 430 comprises a controller 450 configured for controlling a stimulation tool 452 (for example, a brain stimulation tool) to apply stimulation at parameters selected by data processor 433, for example, in response to input by the operator. Stimulation tool 452 can comprise one or more electrodes 454 having a plurality of electrode contacts 456, as further detailed herein. In some embodiments of the present invention, EG system 424, processor 433 and controller 450 operate in a closed loop, wherein processor 433 determine the location and therapeutic effect of the contacts, based on the data from system 424, and wherein controller 450 adjusts the parameters of the treatment of tool 452 responsively to the estimation.

As used herein the term “about” refers to ±10%.

The word “exemplary” is used herein to mean “serving as an example, instance or illustration.” Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments.” Any particular embodiment of the invention may include a plurality of “optional” features unless such features conflict.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Example 1

### Optimization of DBS in Subthalamic Nucleus and Globus Pallidus Among Patients with Parkinson's Disease and Dystonia Using EEG

While the positive effects that DBS of the subthalamic nucleus (STN) and Globus Pallidus (GP) exerts on motor functions in patients with Parkinson's disease (PD) and Dystonia are known, not all patients respond similarly to the treatment. Without wishing to be bound to any particular theory, it is believed that this variability in response is due, at least in part, to the numerous combinations between the chosen DBS electrode contacts and programming settings that include voltage, pulse width and frequency. Traditionally, finding the setting for optimal symptom control requires, on average, about 6 sessions spanned over a period of about 6 months.

In this Example, a technique method for objective differentiation between the locations of four DBS contacts in the various parts of the STN and GP (e.g., the internal segment of the globus pallidus) is described. The differentiation described in this example is based on patterns extracted from EEG recordings. This technique can be used for selecting, optionally and preferably automatically, a subject-specific set of parameters for the neurostimulator of the DBS system. The technique can also be used to estimate the DBS effectiveness as a function of one or more parameters, including, without limitation, the intensity of the DBS stimulus, the frequency of the DBS activation, the band width of the stimulation, the directionality of the stimulation (e.g., within 90° or 180° or 270° or 360°) and the like.

### Protocol

64 to 128 channel EEG was recorded from DBS treated PD patients. Low-frequency stimulation at 2-8 Hz was applied to each of four DBS electrode contacts, individually for several minutes with one minute pause (stimulation OFF) between them. A total of 2000-2400 EEG epochs, aligned to stimulation onset, were averaged to produce a DBS Evoked Response per DBS electrode. In this example, two sets of four electrode contacts were employed. Electrode contacts 0 to 3 at the left side of the brain, and electrode contacts 8 to 11 at the right side of the brain, where electrode contacts 0 and 8 are the most ventral DBS electrode contacts, and electrode contacts 3 and 11 are the most dorsal DBS electrode contacts.

### Data Pre-Processing

Pre-processing was applied to the recorded EEG signals to remove, at least in part, noise due to patient movements, lack of adequate connection between the and the scalp, high power line potentials captured by the EEG electrodes. The pre-processing included the following operations.

Identification of stimulation triggers. Evoked response (ERP) analyses are ones that use time locked repetitive averaged signal in order to extract brain activity. In DBS, these patterns are locked to the stimulation pulse. In this example, the stimulation pulses onsets were extracted from the EEG data based on shapes and patterns of artifacts in the data.

Removal or partial removal of DC shifts. This operation was applied to bring the entire signal to the same mean value.

Filtering. In this example, a bandpass filter was used to remove low and high frequency interferences which are not generated by the brain. The bandpass filter used in this example is characterized a low frequency cutoff of about 0.5 Hz and a high frequency cutoff of about 40 Hz.

Application of Independent Component Analysis (ICA) for removing eye artifacts, cause by, for example, eye movement and eye blinks.

### ERP Analysis

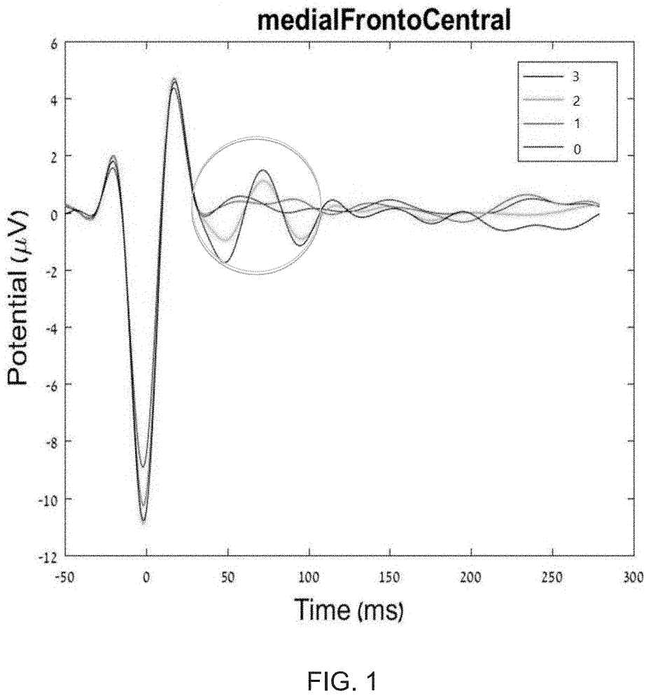

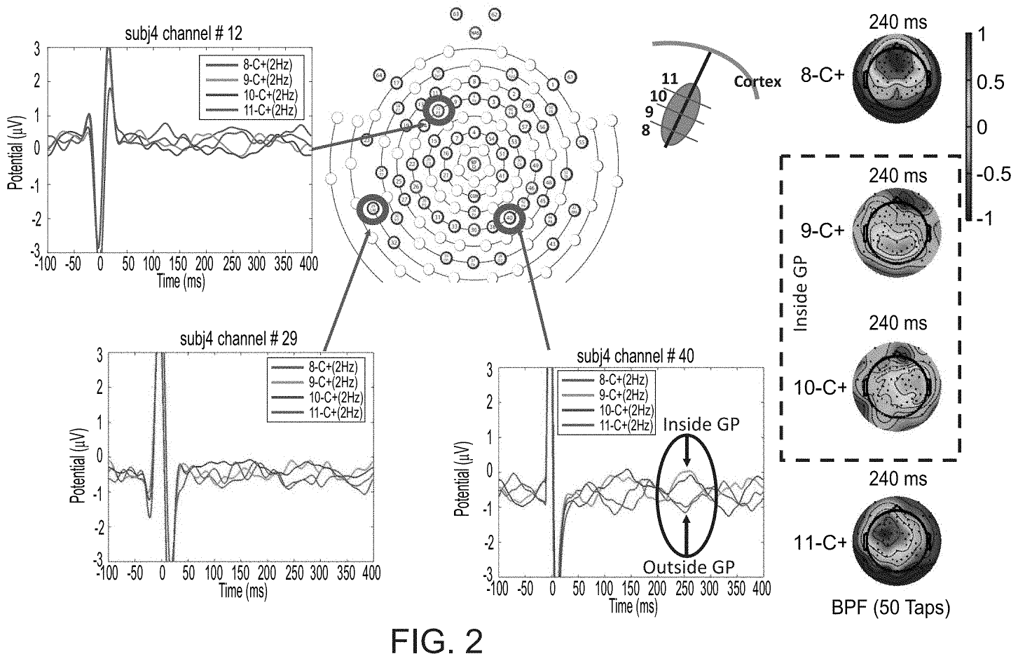

Stimulation at 2-8 Hz was applied for 5-15 minutes (about 2000 trial repetitions) for each of the four DBS electrode contacts 0 to 3 at the left side of the brain, individually with pauses (stimulation OFF) for 1 min between stimulation sessions. For each session, the trials were averaged. All 4 averaged signals were plotted on the same graph as seen in FIG. 1, below, for the STN and in FIG. 2, below, for the GP. FIGS. 1 and 2 show good indication for discrimination between DBS electrode contacts inside and outside the STN and the GP (at about 75 ms and about 240 ms, respectively) after stimulation.

The absolute value of Area Under the Curve was calculated for a predefined time-window of from about 50 to about 100 ms after stimulation onset in STN, which significantly differentiated between the most dorsal DBS electrode contact (in most cases located above the STN, in the zona incerta) and the two ventral contacts in the medial frontal central scalp area (F=5.1, p<0.01, 1-way ANOVA; p<0.02 and 0.03 for 3 vs. 0 and 3 vs. 1, respectively).

The 4 DBS electors (0-3) ERP above described an average ERP of the medial frontal central scalp area as a representative region of interest (ROI) of a representative subject. The analysis was performed for every EEG electrode and amplitudes at specific time-points were extracted from every electrode ERP waveform.

### Scalp Topography Analysis (Topoplot)

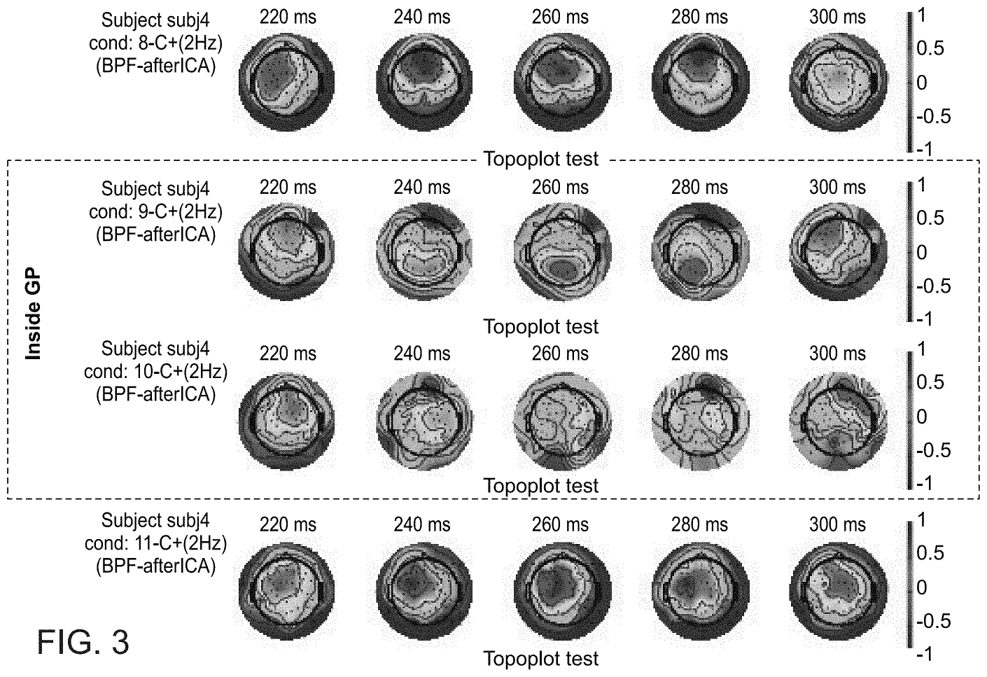

The ERP analysis was performed for all EEG electrodes. Amplitudes at specific time-points were extracted from each ERP waveform of each EEG electrode. The resulted values were interpolated over a spherical surface with regard to each EEG electrode location and plotted (see FIG. 3). The scalp topography gives a spatial discrimination between DBS stimulation inside or outside the GP at the later times of abut 240 ms. In FIG. 3, each row represents a different DBS electrode, the X axis represents time in ms, and the colors represent activation (μV).

### Spatiotemporal Parcellation (STEP) Analysis

### Classification of Therapeutic DBS Contact Using STEP Algorithm and Clustering Methods

When activating each of the 4 DBS electrode contacts separately with some constant stimulation frequency, the ERP exhibits a different time domain pattern for each DBS electrode. With specific reference to the results shown in FIG. 3, stimulations at contacts 8 and 11 result in very similar time domain patterns. When looking at time 240 ms after the DBS pulse stimulation, a very similar topographic pattern is observed.

In the present Example, the DBS electrode contact(s) that has a therapeutic effect is identified automatically from the EEG data. This is optionally and preferably performed based on a parceling procedure that defines spatiotemporal capsules from activity-related features in the EEG data. A procedure suitable for the present embodiment is found in International Publication No. WO2014/076698 the contents of which are hereby incorporated by reference.

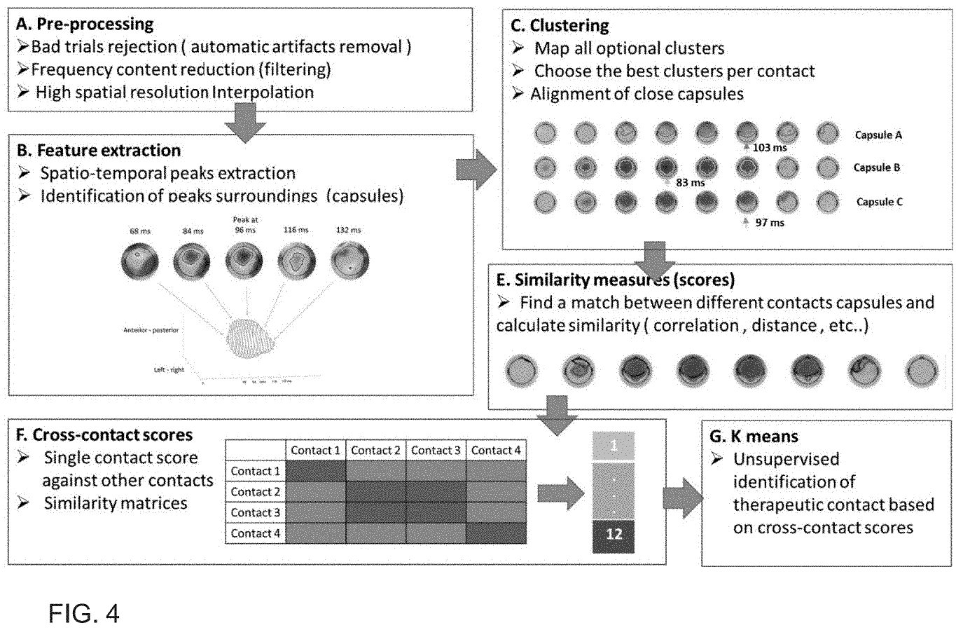

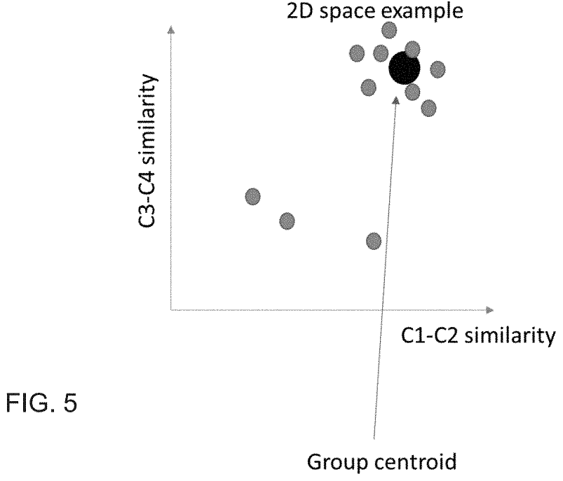

The procedure employed in this example is detailed in Table 1, and FIGS. 4 and 5.

TABLE 1

| | Stage | Process |
|---|---|---|
| 1 | Preprocessing | Automatic preprocessing of raw data acquired |
| | | during stimulation of a single contact. The |
| | | preprocessing is optionally and preferably |
| | | fully automated and includes at least one of: |
| | | filtering, de-trending, automatic artifact |
| | | detection and removal, bad epochs/channel |
| | | detection and removal and average reference. |
| 2 | Feature extraction | Search for spatiotemporal peaks and their |
| | | surroundings to generate spatiotemporal |
| | | structures, e.g., capsules. |
| 3 | Clustering | Choose capsules for contact classification. |
| | | Find close capsules and the capsules they |
| | | match in other contacts. Capsules with no |
| | | match are optionally and preferably discarded. |
| 4 | Similarity measures | Calculate similarity measures according to |
| | (scores) | spatiotemporal parameters of the capsules. |
| 5 | Cross-contact scores | Build multidimensional vectors with the cross- |
| | | contact similarity scores values. Optionally |
| | | and preferably, 6 dimensional or 12 |
| | | dimensional vectors are selected according to |
| | | the data directivity. |
| 6 | Clustering | Find clusters of capsules using a clustering |
| | | algorithm (e.g., k-means or any other |
| | | clustering algorithm), and use the clusters |
| | | for identifying therapeutic contacts. |
| | | Optionally and preferably, the largest clusters |
| | | are selected. |

In parceling procedure finds spatiotemporal structures in the ERP pattern. When performed over the ERP of each of the contacts, it can find similar patterns, which yield similar-activation contacts. By building the cross-contact scores, contacts that excite similar brain activation can be found, and the contact that is optimal for the desired therapy can be identified.

FIG. 4 illustrates a STeP process composed of 6 parts, where steps (a) through (e) involves STeP (capsules) scores generation, and steps (f) and (g) are responsible of automatic classifying of contacts placed within the STN.

The clustering of the STeP results, according to some embodiments of the present invention, optionally and preferably employs K means clustering, more preferably unsupervised K means clustering, but any clustering method can be applied. The matrix results are projected on a multidimensional space (12 dimensions in the present example). Significant values can then be extracted and projected on a smaller multidimensional space. For example, the values can be projected on a six-dimensional space wherein the axis of each dimension measures the level of similarity between two of the four contacts. The clustering method can identify therapeutic contacts by finding similarity between capsules obtained for these contacts. The clustering method can optionally and preferably find a cluster of capsules which represent high similarity in some dimensions and low similarity in the others dimension. Such finding can be indicative that the contacts for which the similarity was higher are therapeutic contacts. The first two axes of a six-dimensional space (similarity between contact Nos. 1 and 2, and similarity between contact Nos. 3 and 4) are shown in FIG. 5.

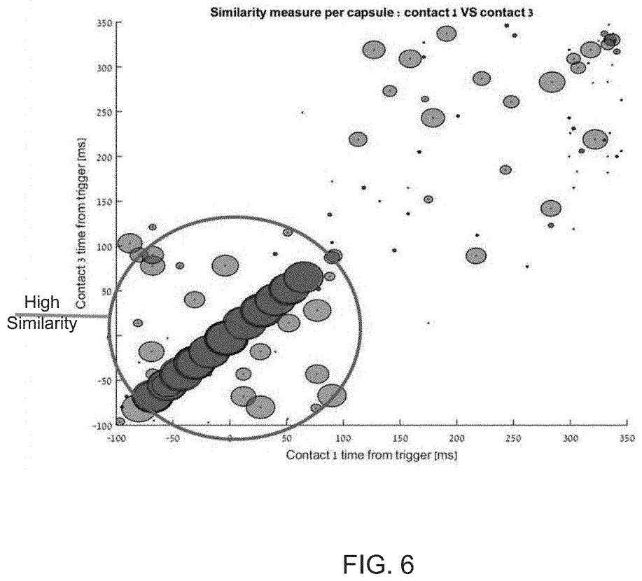

FIG. 6 shows example of a two-dimensional similarity measure for two contacts. Each of the axes is the times of a different contact, each scatter point is a capsule match between two contacts, and the radius of the circle around the scatter point represents the similarity measure between the matched capsules. Shown is the similarity measure for contact Nos. 1 and 3. The large circle shows the time around the artifact (time 0), where very similar artifacts were measured for the two contacts.

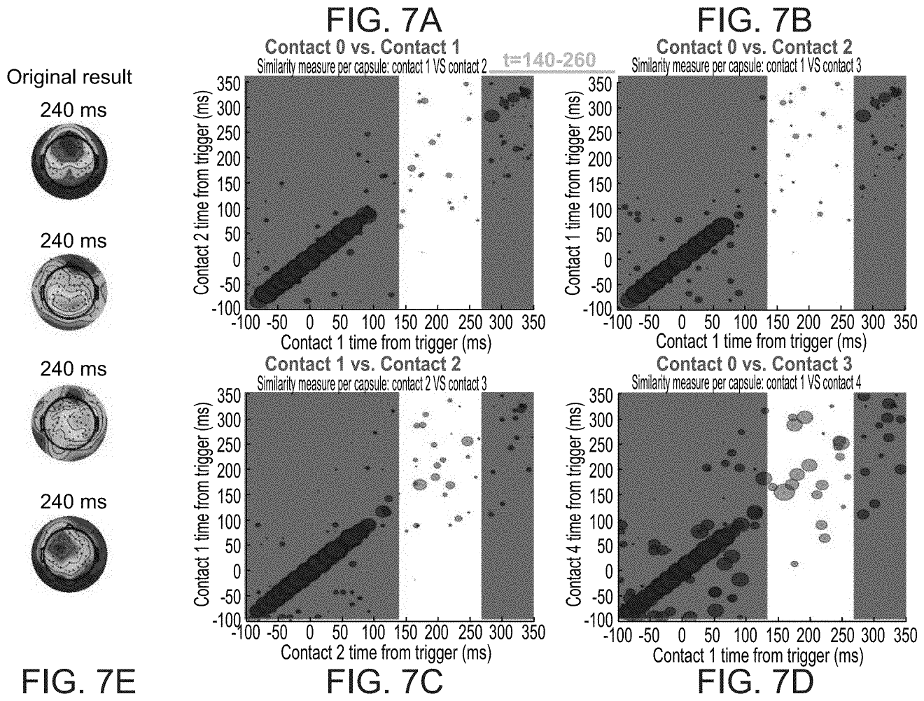

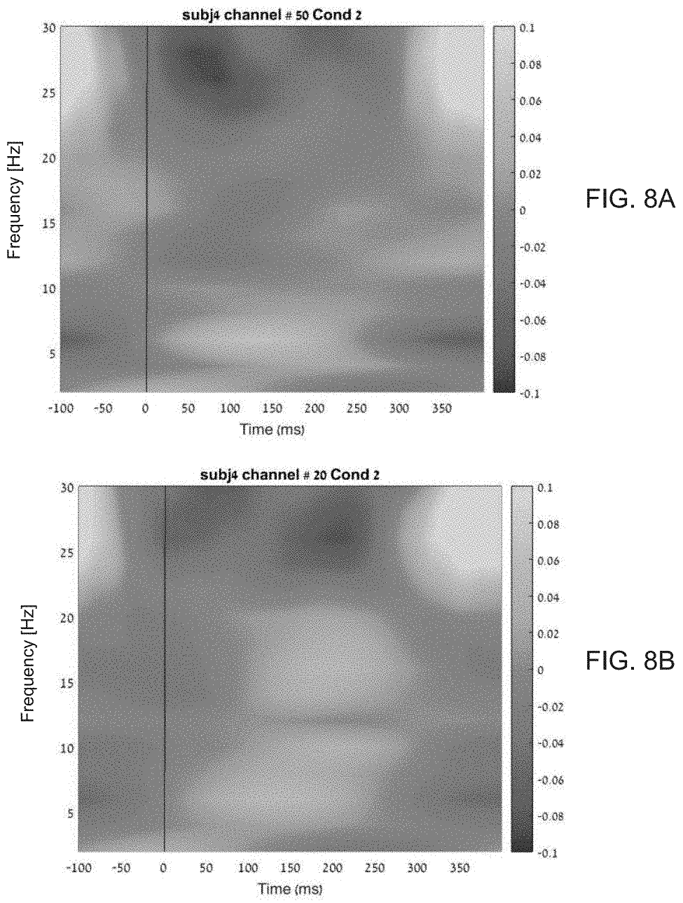

FIGS. 7A-D show two-dimensional similarity measures for contact Nos. 0 and 1 (FIG. 7A), contact Nos. 0 and 2 (FIG. 7B), contact Nos. 1 and 2 (FIG. 7C), and contact Nos. 0 and 3 (FIG. 7D). The obtained capsules at t=240 ms are shown in FIG. 7E. The bright regions in FIGS. 7A-D correspond to a time window of 140-260 ms. As shown, within the time window of 140-260 ms, there is a high similarity between contact Nos. 0 and 3 and also between contact Nos. 1 and 2, since these pairs share many capsules with high similarity measure. On the other hand, there is a lower similarity between contact Nos. 0 and 1 and also between contact Nos. 0 and 2. The method can thus conclude, for example, that contact Nos. 1 and 2 (FIG. 7C) are similar contacts, and also that contact Nos. 0 and 3 (FIG. 7D), are similar contacts.

This findings matches the a priori knowledge that contact Nos. 1 and 2 are inside the STN.

### Event Related Spectral Dynamics (ERSP), Tune Frequency Analysis

ERSP analysis allows discriminating between inside and outside contacts on time-frequency domain. The analysis filters the original ongoing signal (for each contact) at frequency bands (e.g., 2 Hz bands) from about 2 Hz to about 30 Hz. For each band, the energy of the envelope was taken and was normalized against the same band energy for the pause period before the stimulation. Then, ERP was generated for the same signal (energy of the envelope of the ongoing time signal) for each frequency band and were plotted one above another. Contacts outside the GP and contacts inside the GP are characterized by different time-frequency patterns.

FIGS. 8A-H show ERSP maps obtained according to some embodiments of the present invention. Shown are maps of the C3 electrode (FIGS. 8A, 8C, 8E and 8F) and the C4 electrode (FIGS. 8B, 8D, 8F and 8H), with 4 contacts per each electrode, where FIGS. 8A and 8B are for contact No. 8-C+, FIGS. 8C and 8D are for contact No. 9-C+, FIGS. 8E and 8F are for contact No. 10-C+, and FIGS. 8G and 8H are for contact No. 11-C+. In each map, the X axis represents the time in ms, the Y axis represents the frequency in Hz and the color represent the normalized energy, where blue and yellow colors correspond to normalized energy that is far from the ongoing signal, and green color corresponds to normalized energy that is close to the ongoing signal.

FIGS. 8A and 8B and similarly FIGS. 8G and 8H demonstrate a change in the beta frequency in contacts outside the GP (8-C+ and 11-C+, in this example).

### Ongoing Analysis-Response to DBS

An optional pre-processing procedure was applied to the recorded EEG signals. The procedure was aimed to remove, at least in part, noise due to patient movements, lack of adequate connection between the electrode and the scalp, high power line potentials captured by the EEG electrodes. The pre-processing included filtering. In this example, a highpass filter was used to remove low frequency interferences which are not generated by the brain. The highpass filter used in this example is characterized a low frequency cutoff of about 0.5 Hz. Additionally, a two notch filter was used to remove frequency interferences which are not generated by the brain. The notch filter used in this example was characterized by cutoff frequencies of about 50 and 80 Hz. The pre-processing also included application of ICA for removing eye artifacts, cause by, for example, eye movement and eye blinks.

High-frequency stimulation at 130 Hz was applied to each of the four DBS contacts individually for one minutes following one minute pause (stimulation OFF) between stimulations. This analysis quantifies the brain response to a train of DBS stimulations. This procedure uses ongoing EEG analyses for estimating the brain change both during and after the DBS stimulation train.

In the present Example, the ongoing analysis calculated the power spectral density (PSD), using Pwelch method during 130 Hz stimulation times and at pause times. FIGS. 9A-I show PSD analysis of one electrode performed on subject No. 4 according to some embodiments of the present invention. FIG. 9A presents the activity before stimulation (baseline), FIGS. 9A-E present the power during 130 Hz stimulation (ON), and FIGS. 9F-I present the power one minute after the stimulation stops (OFF). FIGS. 9B and 9F correspond to electrode contact No. 8, FIGS. 9C and 9G correspond to electrode contact No. 9, FIGS. 9D and 9H correspond to electrode contact No. 10, and FIGS. 9E and 9I correspond to electrode contact No. 11. Contacts 9 and 10 are inside the right GP. The x axis shows the frequency in Hz, and the y axis is the power in μV²/Hz.

Comparing the PSD both during and after the stimulation allows discrimination between contacts inside or outside the STN or GP.

Specific frequency bands (for example, from about 0.5 Hz to about 80 Hz) can also be extracted, optionally and preferably for each electrode. These bands can be plotted as scalp topography maps to provide spatial distribution of the energy. FIGS. 10A-I show scalp topography maps of the PSD analyses, using Pwelch method during 130 Hz stimulation times and at pause times, for beta range of from about 12 Hz to about 20 Hz, performed according to some embodiments of the present invention. FIG. 10A is the map before stimulation, FIGS. 10B, 10D, 10F and 10H are the maps during 130 Hz stimulation (ON), and FIGS. 10C, 10E, 10G and 10I are the maps during pause (OFF). FIGS. 10B and 10C correspond to electrode contact No. 8, FIGS. 10D and 10E correspond to electrode contact No. 9, FIGS. 10F and 10G correspond to electrode contact No. 10, and FIGS. 10H and 10I correspond to electrode contact No. 11.

### Example 2

### Optimization of DBS in Subthalamic Nucleus Among Patients with Parkinson's Disease Using EEG

The performance of a DBS electrode system has been analyzed for seven Parkinson's disease patients treated by DBS, in accordance with some embodiments of the present invention.

### Method

Scalp EEG was recorded using 128-channels

Low-frequency stimulation was applied at 2-5 Hz, to each of the four DBS electrode contacts. The electrode contacts were numbered 0 for most ventral to 3 for most dorsal.

A total of 2000-2400 EEG epochs were collected. The EEG epochs were aligned to stimulation onset, and were averaged to produce a DBS evoked response per electrode contact.

The medial fronto-central region was defined as the region-of-interest (ROI) for each patient.

An absolute area under the curve was calculated for the ROI over a time-window of 50-100 ms post-stimulation.

### Results

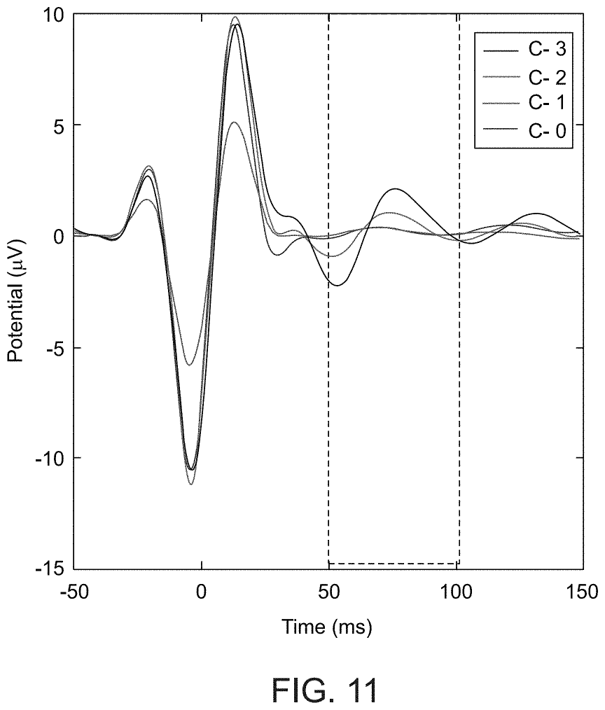

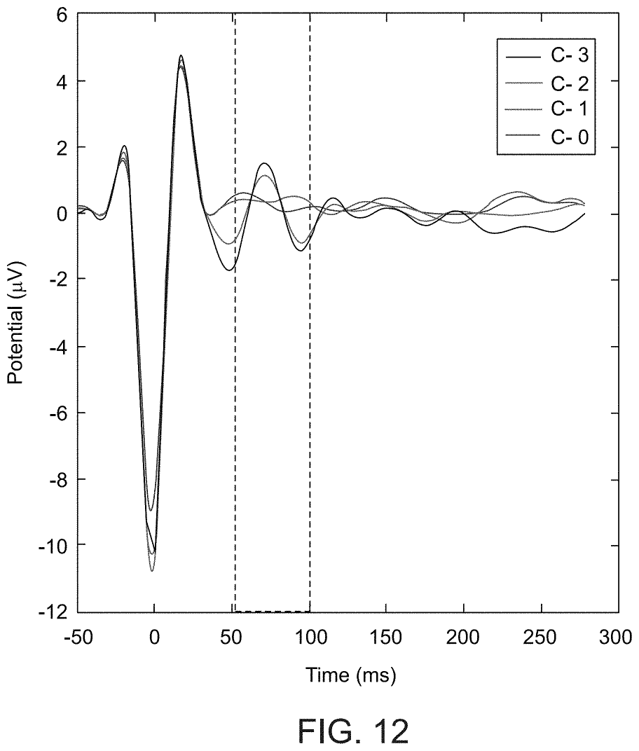

FIGS. 11 and 12 show the potential in μV as a function of the time for the selected ROI, as obtained for each of the four electrode contacts, at stimulation frequency of 5 Hz (FIG. 11) and 2 Hz (FIG. 12). The time-window of 50-100 ms post-stimulation is marked by dashed line. FIGS. 11 and 12 demonstrate that the DBS evoked responses successfully discriminate between the most dorsal DBS electrode contact (which, in the present example was located above the STN, in the zona incerta) and the two ventral contacts, within the 50-100 ms post-stimulation time-window.

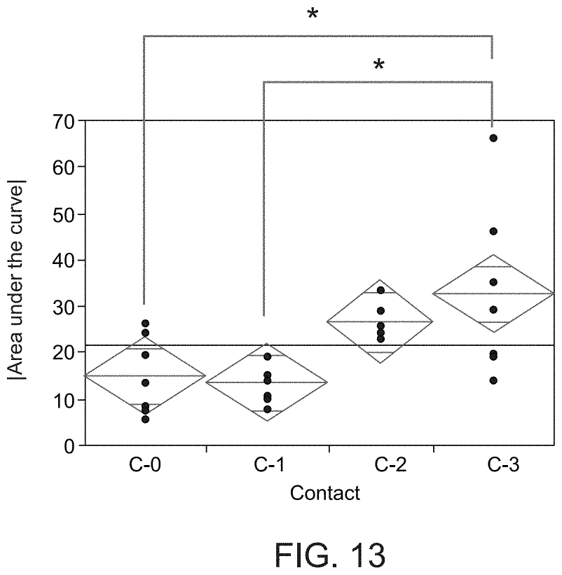

FIG. 13 shows a one-way analysis of variance of the area under the curve as calculated for each of the seven patients. FIG. 13 demonstrates that the DBS evoked response significantly differentiated between the most dorsal DBS electrode contact and the two ventral contacts in the medial frontal central scalp area (F=5.1, (**) p<0.01). Comparisons for all pairs using Tukey-Kramer HSD resulted in (*) p<0.02 for contact No. 3 versus contact No. 0, (*) p<0.03 and for contact No. 3 versus contact No. 1.

### Annex 1

### Spatiotemporal Analysis by Means of a Brain Network Activity (BNA) Pattern

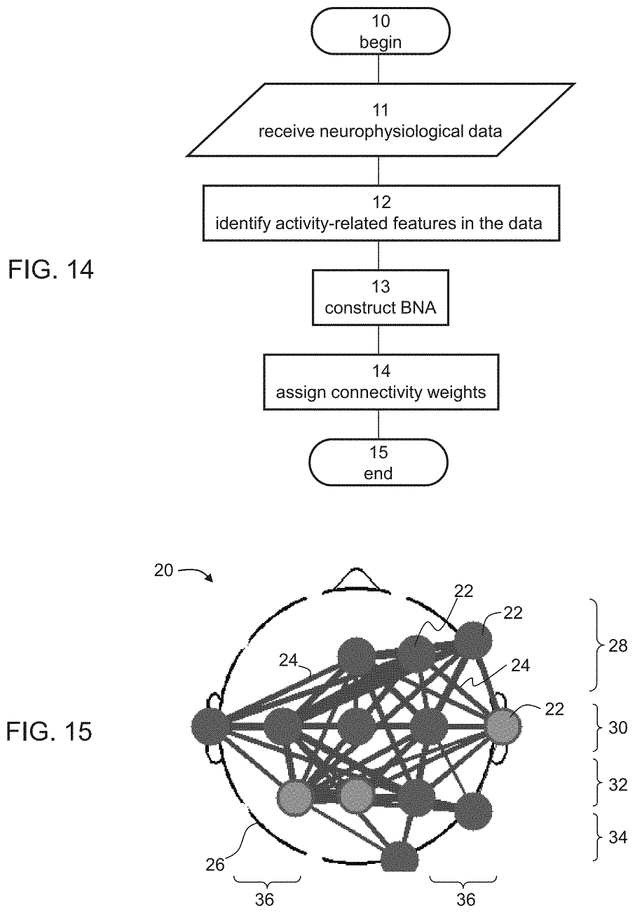

FIG. 14 is a flowchart diagram of a method suitable for analyzing neurophysiological data, according to various exemplary embodiments of the present invention.

The neurophysiological data to be analyzed can be any data acquired directly from the brain of the subject under investigation. The data acquired “directly” in the sense that it shows electrical, magnetic, chemical or structural features of the brain tissue itself. The neurophysiological data can be data acquired directly from the brain of a single subject or data acquired directly from multiple brains of respective multiple subjects (e.g., a research group), not necessarily simultaneously.

Analysis of data from multiple brains can be done by performing the operations described below separately for each portion of the data that correspond to a single brain. Yet, some operations can be performed collectively for more than one brain. Thus, unless explicitly state otherwise, a reference to “subject” or “brain” in the singular form does not necessarily mean analysis of data of an individual subject. A reference to “subject” or “brain” in the singular form encompasses also analysis of a data portion which corresponds to one out of several subjects, which analysis can be applied to other portions as well.

The data can be analyzed immediately after acquisition (“online analysis”), or it can be recorded and stored and thereafter analyzed (“offline analysis”).

Representative example of neurophysiological data types suitable for the present invention, including, without limitation, electroencephalogram (EEG) data, and magnetoencephalography (MEG) data. Optionally, the data include combination of two or more different types of data.

In various exemplary embodiments of the invention the neurophysiological data are associated with signals collected using a plurality of measuring devices respectively placed at a plurality of different locations on the scalp of the subject. In these embodiments, the data type is preferably EEG or MEG data. The measuring devices can include electrodes, superconducting quantum interference devices (SQUIDs), and the like. The portion of the data that is acquired at each such location is also referred to as “channel.” In some embodiments, the neurophysiological data are associated with signals collected using a plurality of measuring devices placed in the brain tissue itself. In these embodiments, the data type is preferably invasive EEG data, also known as electrocorticography (ECoG) data.

Referring now to FIG. 14, the method begins at 10 and optionally and preferably continues to 11 at which the neurophysiological data are received. The data can be recorded directly from the subject or it can be received from an external source, such as a computer readable memory medium on which the data are stored.

The method continues to 12 at which relations between features of the data are determined so as to identify activity-related features. This can be done using any procedure known in the art. For example, procedures as described in International Publication Nos. WO 2007/138579, WO 2009/069134, WO 2009/069135 and WO 2009/069136, the contents of which are hereby incorporated by reference, can be employed. Broadly speaking, the extraction of activity-related features includes multidimensional analysis of the data, wherein the data is analyzed to extract spatial and non-spatial characteristics of the data.

The spatial characteristics preferably describe the locations from which the respective data were acquired. For example, the spatial characteristics can include the locations of the measuring devices (e.g., electrode, SQUID) on the scalp of the subject.

Also contemplated are embodiments in which the spatial characteristics estimate the locations within the brain tissue at which the neurophysiological data were generated. In these embodiments, a source localization procedure, which may include, for example, low resolution electromagnetic tomography (LORETA), is employed. A source localization procedure suitable for the present embodiments is described in the aforementioned international publications which are incorporated by reference. Other source localization procedure suitable for the present embodiments are found in Greenblatt et al., 2005, “Local Linear Estimators for the Bioelectromagnetic Inverse Problem,” IEEE Trans. Signal Processing, 53(9):5430; Sekihara et al., “Adaptive Spatial Filters for Electromagnetic Brain Imaging (Series in Biomedical Engineering),” Springer, 2008; and Sekihara et al., 2005, “Localization bias and spatial resolution of adaptive and non-adaptive spatial filters for MEG source reconstruction,” NeuroImage 25:1056; the contents of which are hereby incorporated by reference.

Additionally contemplated are embodiments in which the spatial characteristics estimate locations on the epicortical surface. In these embodiments, data collected at locations on the scalp of the subject are processed so as to map the scalp potential distribution onto the epicortical surface. The technique for such mapping is known in the art and referred to in the literature as Cortical Potential Imaging (CPI) or Cortical Source Density (CSD). Mapping techniques suitable for the present embodiments are found in Kayser et al., 2006, “Principal Components Analysis of Laplacian Waveforms as a Generic Method for Identifying ERP Generator Patterns: I. Evaluation with Auditory Oddball Tasks,” Clinical Neurophysiology 117(2):348; Zhang et al., 2006, “A Cortical Potential Imaging Study from Simultaneous Extra- and Intra-cranial Electrical Recordings by Means of the Finite Element Method,” Neuroimage, 31(4): 1513; Perrin et al., 1987, “Scalp Current Density Mapping: Value and Estimation from Potential Data,” IEEE transactions on biomedical engineering, BME-34(4):283; Ferree et al., 2000, “Theory and Calculation of the Scalp Surface Laplacian,” www.csi.uoregon.edu/members/ferree/tutorials/SurfaceLaplacian; and Babiloni et al., 1997, “High resolution EEG: a new model-dependent spatial deblurring method using a realistically-shaped MR-constructed subject's head model,” Electroencephalography and clinical Neurophysiology 102:69.

In any of the above embodiments, the spatial characteristics can be represented using a discrete or continuous spatial coordinate system, as desired. When the coordinate system is discrete, it typically corresponds to the locations of the measuring devices (e.g., locations on the scalp, epicortical surface, cerebral cortex or deeper in the brain). When the coordinate system is continuous, it preferably describes the approximate shape of the scalp or epicortical surface, or some sampled version thereof. A sampled surface can be represented by a point-cloud which is a set of points in a three-dimensional space, and which is sufficient for describing the topology of the surface. For a continuous coordinate system, the spatial characteristics can be obtained by piecewise interpolation between the locations of the measuring devices. The piecewise interpolation preferably utilizes a smooth analytical function or a set of smooth analytical functions over the surface.

In some embodiments of the invention the non-spatial characteristics are obtained separately for each spatial characteristic. For example, the non-spatial characteristics can be obtained separately for each channel. When the spatial characteristics are continuous, the non-spatial characteristics are preferably obtained for a set of discrete points over the continuum. Typically, this set of discrete points includes at least the points used for the piecewise interpolation, but may also include other points over the sampled version of the surface.

The non-spatial characteristics preferably include temporal characteristics, which are obtained by segmenting the data according to the time of acquisition. The segmentation results in a plurality of data segments each corresponding to an epoch over which the respective data segment was acquired. The length of the epoch depends on the temporal resolution characterizing the type of neurophysiological data. For example, for EEG or MEG data, a typical epoch length is approximately 1000 ms.

Other non-spatial characteristics can be obtained by data decomposing techniques. In various exemplary embodiments of the invention the decomposition is performed separately for each data segment of each spatial characteristic. Thus, for a particular data channel, decomposition is applied, e.g., sequentially to each data segment of this particular channel (e.g., first to the segment that corresponds to the first epoch, then to the segment that correspond to the second epoch and so on). Such sequential decomposition is performed for other channels as well.

The neurophysiological data can be decomposed by identifying a pattern of extrema (peaks, troughs, etc.) in the data, or, more preferably by means of waveform analysis, such as, but not limited to, wavelet analysis. In some embodiments of the present invention the extremum identification is accompanied by a definition of a spatiotemporal neighborhood of the extremum. The neighborhood can be defined as a spatial region (two- or three-dimensional) in which the extremum is located and/or a time-interval during which the extremum occurs. Preferably, both a spatial region and time-interval are defined, so as to associate a spatiotemporal neighborhood for each extremum. The advantage of defining such neighborhoods is that they provide information regarding the spreading structure of the data over time and/or space. The size of the neighborhood (in terms of the respective dimension) can be determined based on the property of the extremum. For example, in some embodiments, the size of the neighborhood equals the full width at half maximum (FWHM) of the extremum. Other definitions of the neighborhood are not excluded from the scope of the present invention.

The waveform analysis is preferably accompanied by filtering (e.g., bandpass filtering) such that the wave is decomposed to a plurality of overlapping sets of signal extrema (e.g., peaks) which together make up the waveform. The filters themselves may optionally be overlapping.

When the neurophysiological data comprise EEG data, one or more of the following frequency bands can be employed during the filtering: delta band (typically from about 1 Hz to about 4 Hz), theta band (typically from about 3 to about 8 Hz), alpha band (typically from about 7 to about 13 Hz), low beta band (typically from about 12 to about 18 Hz), beta band (typically from about 17 to about 23 Hz), and high beta band (typically from about 22 to about 30 Hz). Higher frequency bands, such as, but not limited to, gamma band (typically from about 30 to about 80 Hz), are also contemplated.

Following the waveform analysis, waveform characteristics, such as, but not limited to, time (latency), frequency and optionally amplitude are preferably extracted. These waveform characteristics are preferably obtained as discrete values, thereby forming a vector whose components are the individual waveform characteristics. Use of discrete values is advantageous since it reduces the amount of data for further analysis. Other reduction techniques, such as, but not limited to, statistical normalization (e.g., by means of standard score, or by employing any statistical moment) are also contemplated. Normalization can be used for reducing noise and is also useful when the method is applied to data acquired from more than one subject and/or when the interfaces between the measuring device and the brain vary among different subjects or among different locations for a single subject. For example, statistical normalization can be useful when there is non-uniform impedance matching among EEG electrodes.

The extraction of characteristics results in a plurality of vectors, each of which includes, as the components of the vector, the spatial characteristics (e.g., the location of the respective electrode or other measuring device), and one or more non-spatial characteristics as obtained from the segmentation and decomposition. Each of these vectors is a feature of the data, and any pair of vectors whose characteristics obey some relation (for example, causal relation wherein the two vectors are consistent with flow of information from the location associated with one vector to the location associated with the other vector) constitutes two activity-related features.

The extracted vectors thus define a multidimensional space. For example, when the components include location, time and frequency, the vectors define a three-dimensional space, and when the components include location, time, frequency and amplitude, the vectors define a four-dimensional space. Higher number of dimensions is not excluded from the scope of the present invention.

When the analysis is applied to neurophysiological data of one subject, each feature of the data is represented as a point within the multidimensional space defined by the vectors, and each set of activity-related features is represented as a set of points such that any point of the set is within a specific distance along the time axis (also referred to hereinbelow as “latency-difference”) from one or more other points in the set.

When the analysis is applied to neurophysiological data acquired from a group or sub-group of subjects, a feature of the data is preferably represented as a cluster of discrete points in the aforementioned multidimensional space. A cluster of points can also be defined when the analysis is applied to neurophysiological data of a single subject. In these embodiments, vectors of waveform characteristics are extracted separately for separate stimuli presented to the subject, thereby defining clusters of points within the multidimensional space, where each point within the cluster corresponds to a response to a stimulus applied at a different time. The separate stimuli optionally and preferably form a set of repetitive presentations of the same or similar stimulus, or a set of stimuli which are not necessarily identical but are of the same type (e.g., a set of not-necessarily identical visual stimuli). Use of different stimuli at different times is not excluded from the scope of the present invention.

Also contemplated are combinations of the above representations, wherein data are collected from a plurality of subjects and for one or more of the subjects, vectors of waveform characteristics are extracted separately for time-separated stimuli (i.e., stimuli applied at separate times). In these embodiments, a cluster contains points that correspond to different subjects as well as points that correspond to a response to a separated stimulus. Consider, for example, a case in which data were collected from 10 subjects, wherein each subject was presented with 5 stimuli during data acquisition. In this case, the dataset includes 5×10=50 data segment, each corresponding to a response of one subject to one stimulus. Thus, in a cluster within the multidimensional space may include up to 5×10 points, each representing a vector of characteristics extracted from one of the data segments.

Whether representing characteristics of a plurality of subjects and/or characteristics of a plurality of responses to stimuli presented to a single subject the width of a cluster along a given axis of the space describes a size of an activity window for the corresponding data characteristic (time, frequency, etc). As a representative example, consider the width of a cluster along the time axis. Such width is optionally and preferably used by the method to describe the latency range within which the event occurs across multiple subjects. Similarly, the width of a cluster along the frequency axis can be used for describing the frequency band indicating an occurrence of an event occurring across multiple subjects; the widths of a cluster along the location axes (e.g., two location axes for data corresponding to a 2D location map, and three location axes for data corresponding to a 3D location map) can be used to define a set of adjoining electrodes at which the event occurs across multiple subjects, and the width of a cluster along the amplitude axis can be used to define an amplitude range indicating an occurrence of event across multiple subjects.

For a group or sub-group of subjects, activity-related features can be identified as follows. A single cluster along the time axis is preferably identified as representing a unitary event occurring within a time window defined, as stated, by the width of the cluster. This window is optionally and preferably narrowed to exclude some outlier points, thereby redefining the latency range characterizing the respective data feature. For a succession of clusters along the time axis, wherein each cluster in the series has a width (along the time axis) within a particular constraint, a pattern extraction procedure is preferably implemented for identifying those clusters which obey connectivity relations thereamongst. Broadly speaking such procedure can search over the clusters for pairs of clusters in which there are connectivity relations between a sufficient number of points between the clusters.

The pattern extraction procedure can include any type of clustering procedures, including, without limitation, a density-based clustering procedure, a nearest-neighbor-based clustering procedure, and the like. A density-based clustering procedure suitable for the present embodiments is described in Cao et al., 2006, “Density-based clustering over an evolving data stream with noise,” Proceedings of the Sixth SIAM International Conference on Data Mining. Bethesda, Md., p. 328-39. A nearest-neighbor clustering procedure suitable for the present embodiments is described in [R. O. Duda, P. E. Hart and D. G. Stork, “Pattern Classification” (2nd Edition), A Wiley-Interscience Publication, 2000]. When nearest-neighbor clustering procedure is employed, clusters are identified and thereafter gathered to form meta-clusters based on spatiotemporal distances among the clusters. The meta-clusters are, therefore, clusters of the identified clusters. In these embodiments, the meta-clusters are the features of the data, and activity-related features are identified among the meta-clusters.

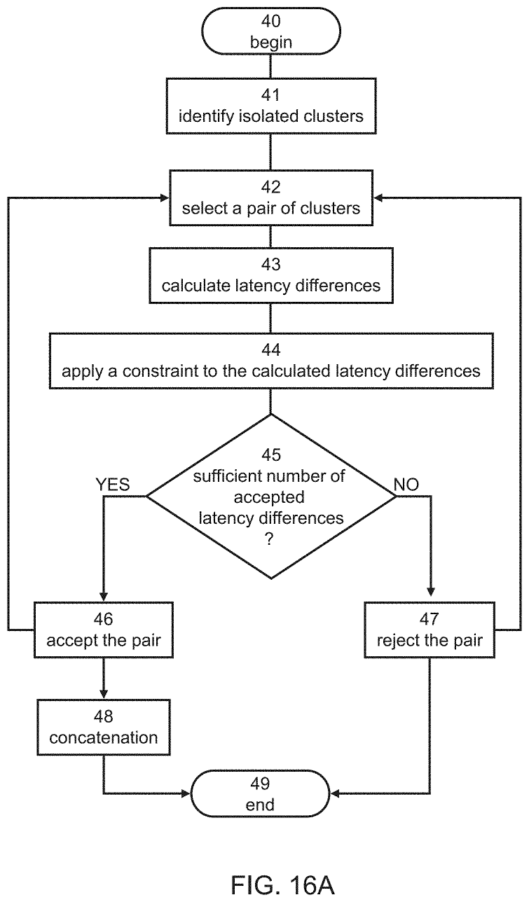

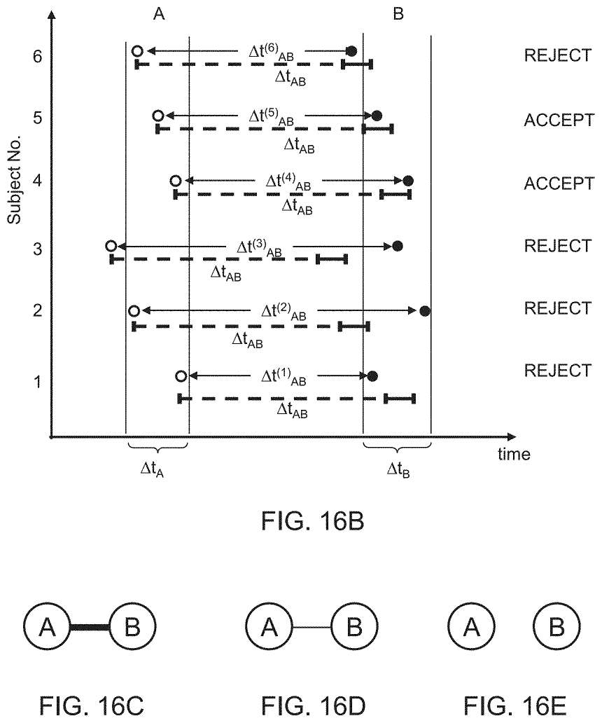

FIG. 16A is a flowchart diagram describing a procedure for identifying activity-related features for a group of subjects, according to some embodiments of the present invention. The procedure begins at 40 and continues to 41 at which isolated clusters are identified. The present embodiments contemplate both subspace clustering, wherein clusters are identified on a particular projection of the multidimensional space, and full-space clustering wherein clusters are identified on the entire multidimensional space. Subspace clustering is preferred from the standpoint of computation time, and full-space clustering is preferred from the standpoint of features generality.

One representative example of subspace clustering includes identification of clusters along the time axis, separately for each predetermined frequency band and each predetermined spatial location. The identification optionally and preferably features a moving time-window with a fixed and predetermined window width. A typical window width for EEG data is about 200 ms for the delta band. A restriction on a minimal number of points in a cluster is optionally applied so as not to exclude small clusters from the analysis. Typically cluster with less than X points, where X equals about 80% of the subjects in the group, are excluded. The minimal number of points can be updated during the procedure. Once an initial set of clusters is defined, the width of the time window is preferably lowered.

Another representative example of subspace clustering includes identification of clusters over a space-time subspace, preferably separately for each predetermined frequency band. In this embodiment, the extracted spatial characteristics are represented using a continuous spatial coordinate system, e.g., by piecewise interpolation between the locations of the measuring devices, as further detailed hereinabove. Thus, each cluster is associated with a time window as well as a spatial region, wherein the spatial region may or may not be centered at a location of a measuring device. In some embodiments, at least one cluster is associated with a spatial region which is centered at a location other than a location of a measuring device. The space-time subspace is typically three-dimensional with one temporal dimension and two spatial dimensions, wherein each cluster is associated with a time-window and a two-dimensional spatial region over a surface which may correspond, e.g., to the shape of the scalp surface, the epicortical surface and the like. Also contemplated is a four-dimensional space-time space wherein each cluster is associated with a time-window and a three-dimensional spatial region over a volume corresponding, at least in part, to internal brain.

Another representative example of subspace clustering includes identification of clusters over a frequency-space-time subspace. In this embodiment, instead of searching for clusters separately for each predetermined frequency band, the method allows identification of clusters also at frequencies which are not predetermined. Thus, the frequency is considered as a continuous coordinate over the subspace. As in the embodiment of space-time subspace, the extracted spatial characteristics are represented using a continuous spatial coordinate system. Thus, each cluster is associated with a time window, a spatial region and a frequency band. The spatial region can be two- or three-dimensional as further detailed hereinabove. In some embodiments, at least one cluster is associated with a spatial region which is centered at a location other than a location of a measuring device, and at least one cluster is associated with a frequency band which includes frequencies of two or more of the delta, theta, alpha, low beta, beta, high beta and gamma bands. For example, a cluster can be associated with a frequency band spanning over part of the delta band and part of the theta band, or part of the theta band and part of the alpha band, or part of the alpha band and part of the low beta band, etc.

The procedure optionally and preferably continues to 42 at which, a pair of clusters is selected. The procedure optionally and preferably continues to 43 at which, for each subject that is represented in the selected pair, latency difference (including zero difference) between the corresponding events is optionally calculated. The procedure continues to 44 at which a constraint is applied to the calculated latency differences such that latency differences which are outside a predetermined threshold range (e.g., 0-30 ms) are rejected while latency differences which are within the predetermined threshold range are accepted. The procedure continues to decision 45 at which the procedure determines whether the number of accepted differences is sufficiently large (i.e., above some number, e.g., above 80% of the subjects in the group). If the number of accepted differences is not sufficiently large the procedure proceeds to 46 at which the procedure accepts the pair of clusters and identifies it as a pair of activity-related features. If the number of accepted differences is sufficiently large the procedure proceeds to 47 at which the procedure reject the pair. From 46 or 47 the procedure of the present embodiments loops back to 42.

An illustrative example for determining relations among the data features and identification of activity-related features is shown in FIG. 16B. The illustration is provided in terms of a projection onto a two-dimensional space which includes time and location. The present example is for an embodiment in which the spatial characteristics are discrete, wherein the identification of clusters is along the time axis, separately for each predetermined frequency band and each predetermined spatial location. The skilled person would know how to adapt the description for the other dimensions, e.g., frequency, amplitude, etc. FIG. 16B illustrates a scenario in which data are collected from 6 subjects (or from a single subject, present with 6 stimuli at different times), enumerated 1 through 6. For clarity of presentation, different data segments data (e.g., data collected from different subjects, or from the same subject but for stimuli of different times) are separated along a vertical axis denoted “Data Segment No.” For each segment, an open circle represents an event recorded at one particular location (by means of a measuring device, e.g., EEG electrode) denoted “A”, and a solid disk represents an event recorded at another particular location denoted “B”.

The time axis represents the latency of the respective event, as measured, e.g., from a time at which the subject was presented with a stimulus. The latencies of the events are denoted herein t⁽ⁱ⁾_{A} and t⁽ⁱ⁾_{B}, where i represents the segment index (i=1, . . . , 6) and A and B represent the location. For clarity of presentation, the latencies are not shown in FIG. 16B, but one of ordinary skills in the art, provided with the details described herein would know how to add the latencies to the drawing.

For each of locations A and B, a time window is defined. These time windows, denoted Δt_{A} and Δt_{B}, correspond to the width of the clusters along the time axis and they can be the same or different from one another, as desired. Also defined is a latency difference window Δt_{AB}, between the two unitary events. This window corresponds to the separation along the time axis between the clusters (e.g., between their centers). The window Δt_{AB} is illustrated as an interval having a dashed segment and a solid segment. The length of the dashed segment represents the lower bound of the window and the overall length of the interval represents the upper bound of the window. Δt_{A}, Δt_{B} and Δt_{AB} are part of the criteria for determining whether to accept the pair of events recorded at A and B as activity-related features.

The time windows Δt_{A} and Δt_{B} are preferably used for identifying unitary events in the group. As shown, for each of segment Nos. 1, 2, 4 and 5 both events fall within the respective time windows (mathematically, this can be written as follows: t⁽ⁱ⁾_{A}∈Δt_{A}, t⁽ⁱ⁾_{B}∈Δt_{A}, i=1, 2, 4, 5). On the other hand, for segment No. 3 the event recorded at A falls outside Δt_{A}(t⁽³⁾_{A}∉Δt_{A}) while the event recoded at B falls within Δt_{B} (t⁽³⁾_{B}∈Δt_{B}), and for segment No. 6 the event recorded at A falls within Δt_{A}(t⁽⁶⁾_{A}∈Δt_{A}) while the event recoded at B falls outside Δt_{B} (t⁽⁶⁾_{B}∉Δt_{B}). Thus, for location A, a unitary event is defined as a cluster of data points obtained from segment Nos. 1, 2, 4, 5 and 6, and for location B, a unitary event is defined as a cluster of data points obtained from segment Nos. 1-5.

The latency difference window Δt_{AB} is preferably used for identifying activity-related features. In various exemplary embodiments of the invention the latency difference Δt⁽ⁱ⁾_{AB} (i=1, 2, . . . , 5) of each segment is compared to the latency difference window Δt_{AB}. In various exemplary embodiments of the invention a pair of features is accepted as an activity-related pair if (i) each of the features in the pair belongs to a unitary event, and (ii) the corresponding latency difference falls within Δt_{AB}. In the illustration of FIG. 16B, each of the pairs recorded from segment Nos. 4 and 5 is accepted as a pair of activity-related features, since both criteria are met for each of those segment (Δt⁽ⁱ⁾A_{B}∈Δt_{AB}, t⁽ⁱ⁾_{A}∈Δt_{A}, t⁽ⁱ⁾_{B}∈Δt_{A}, i=4, 5). The pairs recorded from segment Nos. 1-3 do not pass the latency difference criterion since each of Δt⁽¹⁾_{AB}, Δt⁽²⁾_{AB} and Δt⁽³⁾_{AB} is outside Δt_{AB} (Δt⁽ⁱ⁾_{AB}∉Δt_{AB}, i=1, 2, 3). These pairs are, therefore, rejected. Notice that in the present embodiment, even though the pair obtained from segment No. 6 passes the latency difference criterion, the pair is rejected since it fails to pass the time-window criterion (Δt⁽⁶⁾_{AB}∉Δt_{AB}).

In various exemplary embodiments of the invention the procedure also accepts pairs corresponding to simultaneous events of the data that occur at two or more different locations. Although such events are not causal with respect to each other (since there is no flow of information between the locations), the corresponding features are marked by the method. Without being bounded to any particular theory, the present inventors consider that simultaneous events of the data are causally related to another event, although not identified by the method. For example, the same physical stimulus can generate simultaneous events in two or more locations in the brain.

The identified pairs of activity-related features, as accepted at 46, can be treated as elementary patterns which can be used as elementary building blocks for constructing complex patterns within the feature space. In various exemplary embodiments of the invention, the method proceeds to 48 at which two or more pairs of activity-related features are joined (e.g., concatenated) to form a pattern of more than two features. The criterion for the concatenation can be similarity between the characteristics of the pairs, as manifested by the vectors. For example, in some embodiments, two pairs of activity-related features are concatenated if they have a common feature. Symbolically, this can be formulated as follows: the pairs “A-B” and “B-C” have “B” as a common feature and are concatenated to form a complex pattern A-B-C. Preferably, the concatenated set of features is subjected to a thresholding procedure, for example, when X % or more of the subjects in the group are included in the concatenated set, the set is accepted, and when less than X % of the subjects in the group are included in the concatenated set, the set is rejected. A typical value for the threshold X is about 80.

Each pattern of three or more features thus corresponds to a collection of clusters defined such that any cluster of the collection is within a specific latency-difference from one or more other clusters in the collection. Once all pairs of clusters are analyzed the procedures continues to terminator 49 at which it ends.

Referring again to FIG. 14, at 13 a brain network activity (BNA) pattern is constructed.

The concept of BNA pattern can be better understood with reference to FIG. 15 which is a representative example of a BNA pattern 20 which may be extracted from neurophysiological data, according to some embodiments of the present invention. BNA pattern 20 has a plurality of nodes 22, each representing one of the activity-related features. For example, a node can represent a particular frequency band (optionally two or more particular frequency bands) at a particular location and within a particular time-window or latency range, optionally with a particular range of amplitudes.

Some of nodes 22 are connected by edges 24 each representing the causal relation between the nodes at the ends of the respective edge. Thus, the BNA pattern is a represented as a graph having nodes and edges. In various exemplary embodiments of the invention the BNA pattern includes plurality of discrete nodes, wherein information pertaining to features of the data is represented only by the nodes and information pertaining to relations among the features is represented only by the edges.

FIG. 15 illustrates BNA pattern 20 within a template 26 of a scalp, allowing relating the location of the nodes to the various lobes of the brain (frontal 28, central 30, parietal 32, occipital 34 and temporal 36). The nodes in the BNA pattern can be labeled by their various characteristics. A color coding or shape coding visualization technique can also be employed, if desired. For example, nodes corresponding to a particular frequency band can be displayed using one color or shape and nodes corresponding to another frequency band can be displayed using another color or shape. In the representative example of FIG. 15, two colors are presented. Red nodes correspond to Delta waves and green nodes correspond to Theta waves.

BNA pattern 20 can describe brain activity of a single subject or a group or sub-group of subjects. A BNA pattern which describes the brain activity of a single subject is referred to herein as a subject-specific BNA pattern, and BNA pattern which describes the brain activity of a group or sub-group of subjects is referred to herein as a group BNA pattern.

When BNA pattern 20 is a subject-specific BNA pattern, only vectors extracted from data of the respective subject are used to construct the BNA pattern. Thus, each node corresponds to a point in the multidimensional space and therefore represents an activity event in the brain. When BNA pattern 20 is a group BNA pattern, some nodes can correspond to a cluster of points in the multidimensional space and therefore represents an activity event which is prevalent in the group or sub-group of subjects. Due to the statistical nature of a group BNA pattern, the number of nodes (referred to herein as the “order”) and/or edges (referred to herein as the “size”) in a group BNA pattern is typically, but not necessarily, larger than the order and/or size of a subject-specific BNA pattern.

As a simple example for constructing a group BNA pattern, the simplified scenario illustrated in FIG. 16B is considered, wherein a “segment” corresponds to a different subject in a group or sub-group of subjects. The group data include, in the present example, two unitary events associated with locations A and B. Each of these events forms a cluster in the multidimensional space. In various exemplary embodiments of the invention each of the clusters, referred to herein as clusters A and B, is represented by a node in the group BNA. The two clusters A and B are identified as activity-related features since there are some individual points within these clusters that pass the criteria for such relation (the pairs of Subject Nos. 4 and 5, in the present example). Thus, in various exemplary embodiments of the invention the nodes corresponding to clusters A and B are connected by an edge. A simplified illustration of the resulting group BNA pattern is illustrated in FIG. 16C.

A subject-specific BNA pattern is optionally and preferably constructed by comparing the features and relations among features of the data collected from the respective subject to the features and relations among features of reference data, which, in some embodiments of the present invention comprise group data. In these embodiments, points and relations among points associated with the subject's data are compared to clusters and relations among clusters associated with the group's data. Consider, for example, the simplified scenario illustrated in FIG. 16B, wherein a “segment” corresponds to a different subject in a group or sub-group of subjects. Cluster A does not include a contribution from Subject No. 3, and cluster B does not include a contribution from Subject No. 6, since for these subjects the respective points fail to pass the time-window criterion. Thus, in various exemplary embodiments of the invention when a subject-specific BNA pattern is constructed for Subject No. 3 it does not include a node corresponding to location A, and when a subject-specific BNA pattern is constructed for Subject No. 6 it does not include a node corresponding to location B. On the other hand, both locations A and B are represented as nodes in the subject-specific BNA patterns constructed for any of Subject Nos. 1, 2, 4 and 5.

For those subjects for which the respective points are accepted as a pair of activity-related features (Subject Nos. 4 and 5, in the present example), the corresponding nodes are preferably connected by an edge. A simplified illustration of a subject-specific BNA pattern for such a case is shown in FIG. 16D.

Note that for this simplified example of only two nodes, the subject-specific BNA of FIG. 16D is similar to the group BNA of FIG. 16C. For a larger number of nodes, the order and/or size of the group BNA pattern is, as stated, typically larger than the order and/or size of the subject-specific BNA pattern. An additional difference between the subject-specific and group BNA patterns can be manifested by the degree of relation between the activity-related features represented by the edges, as further detailed hereinbelow.

For subjects for which the respective points were rejected (Subject Nos. 1 and 2, in the present example), the corresponding nodes are preferably not connected by an edge. A simplified illustration of a subject-specific BNA pattern for such case is shown in FIG. 16E.

It is to be understood, however, that although the above technique for constructing a subject-specific BNA pattern is described in terms of the relation between the data of a particular subject to the data of a group of subjects, this need not necessarily be the case, since in some embodiments, a subject-specific BNA pattern can be constructed only from the data of a single subject. In these embodiments, vectors of waveform characteristics are extracted separately for time-separated stimuli, to define clusters of points where each point within the cluster corresponds to a response to a stimulus applied at a different time, as further detailed hereinabove. The procedure for constructing subject-specific BNA pattern in these embodiments is preferably the same as procedure for constructing a group BNA pattern described above. However, since all data are collected from a single subject, the BNA pattern is subject-specific.

Thus, the present embodiments contemplate two types of subject-specific BNA patterns: a first type that describes the association of the particular subject to a group or sub-group of subjects, which is a manifestation of a group BNA pattern for the specific subject, and a second type that describes the data of the particular subject without associating the subject to a group or sub-group of subjects. The former type of BNA pattern is referred to herein as an associated subject-specific BNA pattern, and the latter type of BNA pattern is referred to herein as an unassociated subject-specific BNA pattern.

For unassociated subject-specific BNA pattern, the analysis is preferably performed on the set of repetitive presentations of a single stimulus, namely on a set of single trials, optionally and preferably before averaging the data and turning it to one single vector of the data. For group BNA patterns, on the other hand, the data of each subject of the group is optionally and preferably averaged and thereafter turned into vectors of the data.

Note that while the unassociated subject-specific BNA pattern is generally unique for a particular subject (at the time the subject-specific BNA pattern is constructed), the same subject may be characterized by more than one associated subject-specific BNA patterns, since a subject may have different associations to different groups. Consider for example a group of healthy subjects and a group of non-healthy subjects all suffering from the same brain disorder. Consider further a subject Y which may or may not belong to one of those groups. The present embodiments contemplate several subject-specific BNA patterns for subject Y. A first BNA pattern is an unassociated subject-specific BNA pattern, which, as stated is generally unique for this subject, since it is constructed from data collected only from subject Y. A second BNA pattern is an associated subject-specific BNA pattern constructed in terms of the relation between the data of a subject Y to the data of the healthy group. A third BNA pattern is an associated subject-specific BNA pattern constructed in terms of the relation between the data of a subject Y to the data of the non-healthy group. Each of these BNA patterns are useful for assessing the condition of subject Y. The first BNA pattern can be useful, for example, for monitoring changes in the brain function of the subject over time (e.g., monitoring brain plasticity or the like) since it allows comparing the BNA pattern to a previously constructed unassociated subject-specific BNA pattern. The second and third BNA pattern can be useful for determining the level of association between subject Y and the respective group, thereby determining the likelihood of brain disorder for the subject.

Also contemplated are embodiments in which the reference data used for constructing the subject-specific BNA pattern corresponds to history data previously acquired from the same subject. These embodiments are similar to the embodiments described above regarding the associated subject-specific BNA pattern, except that the BNA pattern is associated to the history of the same subject instead of to a group of subjects.

Additionally contemplated are embodiments in which the reference data corresponds to data acquired from the same subject at some later time. These embodiments allow investigating whether data acquired at an early time evolve into the data acquired at the later time. A particular and non limiting example is the case of several treatment sessions, e.g., N sessions, for the same subject. Data acquired in the first several treatment sessions (e.g., from session 1 to session k₁<N) can be used as reference data for constructing a first associated subject-specific BNA pattern corresponding to mid sessions (e.g., from session k₂>k₁ to session k₃>k₂), and data acquired in the last several treatment sessions (e.g., from session k₄ to session N) can be used as reference data for constructing a second associated subject-specific BNA pattern corresponding to the aforementioned mid sessions, where 1<k₁<k₂<k₃<k₄. Such two associated subject-specific BNA patterns for the same subject can be used for determining data evolution from the early stages of the treatment to the late stages of the treatment.

The method proceeds to 14 at which a connectivity weight is assigned to each pair of nodes in the BNA pattern (or, equivalently, to each edge in the BNA) pattern, thereby providing a weighted BNA pattern. The connectivity weight is represented in FIGS. 12, 13C and 13D by the thickness of the edges connecting two nodes. For example, thicker edges can correspond to higher weights and thinner edges can correspond to lower weights.

In various exemplary embodiments of the invention the connectivity weight comprises a weight index WI calculated based on at least one of the following cluster properties: (i) the number of subjects participating in the corresponding cluster pair, wherein greater weights are assigned for larger number of subjects; (ii) the difference between the number of subjects in each cluster of the pair (referred to as the “differentiation level” of the pair), wherein greater weights are assigned for lower differentiation levels; (iii) the width of the time windows associated with each of the corresponding clusters (see, e.g., Δt_{A} and Δt_{B} in FIG. 16A), wherein greater weights are assigned for narrower windows; (iv) the latency difference between the two clusters (see Δt_{AB} in FIG. 16A), wherein greater weights are assigned for narrower windows; (v) the amplitude of the signal associated with the corresponding clusters; (vi) the frequency of the signal associated with the corresponding clusters; and (vii) the width of a spatial window defining the cluster (in embodiments in which the coordinate system is continuous). For any of the cluster properties, except properties (i) and (ii), one or more statistical observables of the property, such as, but not limited to, average, median, supremum, infimum and variance over the cluster are preferably used.

For a group BNA pattern or an unassociated subject-specific BNA pattern, the connectivity weight preferably equals the weight index WI as calculated based on the cluster properties.

For an associated subject-specific BNA pattern, the connectivity weight of a pair of nodes is preferably assigned based on the weight index WI as well as one or more subject-specific and pair-specific quantities denoted SI. Representative examples of such quantities are provided below.

In various exemplary embodiments of the invention a pair of nodes of the associated subject-specific BNA pattern is assigned with a connectivity weight which is calculated by combining WI with SI. For example, the connectivity weight of a pair in the associated subject-specific BNA pattern can be given by WI·SI. When more than one quantities (say N quantities) are calculated for a given pair of nodes, the pair can be assigned with more than one connectivity weights, e.g., WI·SI₁, WF·SI₂, . . . , WI·SI_{N}, wherein SI₁, SI₂, . . . , SI_{N}, are N calculated quantities. Alternatively or additionally, all connectivity weights of a given pair can be combined, e.g., by averaging, multiplying and the like.

The quantity SI can be, for example, a statistical score characterizing the relation between the subject-specific pair and the corresponding clusters. The statistical score can be of any type, including, without limitation, deviation from average, absolute deviation, standard-score and the like. The relation for whom the statistical score is calculated can pertain to one or more properties used for calculating the weight index WI, including, without limitation, latency, latency difference, amplitude, frequency and the like.

A statistical score pertaining to latency or latency difference is referred to herein as a synchronization score and denoted SIs. Thus, a synchronization score according to some embodiments of the present invention can be obtained by calculating a statistical score for (i) the latency of the point as obtained for the subject (e.g., t⁽ⁱ⁾_{A} and t⁽ⁱ⁾_{B}, in the above example) relative to the group-average latency of the corresponding cluster, and/or (ii) the latency difference between two points as obtained for the subject (e.g., Δt⁽ⁱ⁾_{AB}), relative to the group-average latency difference between the two corresponding clusters.

A statistical score pertaining to amplitude is referred to herein as an amplitude score and denoted SIa. Thus an amplitude score according to some embodiments of the present invention is obtained by calculating a statistical score for the amplitude as obtained for the subject relative to the group-average amplitude of the corresponding cluster.

A statistical score pertaining to frequency is referred to herein as a frequency score and denoted SIf. Thus a frequency score according to some embodiments of the present invention is obtained by calculating a statistical score for the frequency as obtained for the subject relative to the group-average frequency of the corresponding cluster.

A statistical score pertaining to the location is referred to herein as a location score and denoted SIl. These embodiments are particularly useful in embodiments in which a continuous coordinate system is employed, as further detailed hereinabove. Thus a location score according to some embodiments of the present invention is obtained by calculating a statistical score for the location as obtained for the subject relative to the group-average location of the corresponding cluster.

Calculation of statistical scores pertaining to other properties is not excluded from the scope of the present invention.

Following is a description of a technique for calculating the quantity SI, according to some embodiments of the present invention.

When SI is a synchronization score SIs the calculation is optionally and preferably based on the discrete time points matching the spatiotemporal constraints set by the electrode pair (Time_{subj}), if such exist. In these embodiments, the times of these points can are compared to the mean and standard deviation of the times of the discrete points participating in the group pattern (Timeₚₐₜ), for each region to provide a regional synchronization score SIsᵣ. The synchronization score SIs can then be calculated, for example, by averaging the regional synchronization scores of the two regions in the pair. Formally, this procedure can be written as:

$${SIs}_{r} = 0.5 + \frac{std ( {Time}_{pat} )}{2 * ( abs ( \overline{{Time}_{pat}} - {Time}_{subj} ) + std ( {Time}_{pat} ) )} ; SIs = \frac{1}{r} Σ {SIs}_{r}$$

An amplitude score SIa, is optionally and preferably calculated in a similar manner. Initially the amplitude of the discrete points of the individual subject (Amp_{subj}) is compared to the mean and standard deviation of the amplitudes of the discrete points participating in the group pattern (Ampₚₐₜ), for each region to provide a regional amplitude score SIaᵣ. The amplitude score can then be calculated, for example, by averaging the regional amplitude scores of the two regions in the pair:

$${SIa}_{r} = 0.5 + \frac{std ( {Amp}_{pat} )}{2 * ( abs ( \overline{{Amp}_{pat}} - {Amp}_{subj} ) + std ( {Amp}_{pat} ) )} ; SIa = \frac{1}{r} Σ {SIa}_{r}$$

One or more BNA pattern similarities S can then be calculated as a weighted average over the nodes of the BNA pattern, as follows:

$$Ss = \frac{{\sum }_{i} ( {W}_{i} * {SIs}_{i} )}{{\sum }_{i} {W}_{i}}$$

$$Sa = \frac{{\sum }_{i} ( {W}_{i} * {SIa}_{i} )}{{\sum }_{i} {W}_{i}}$$

$$Sf = \frac{{\sum }_{i} ( {W}_{i} * {SIf}_{i} )}{{\sum }_{i} {W}_{i}}$$

$$Sl = \frac{{\sum }_{i} ( {W}_{i} * {SIl}_{i} )}{{\sum }_{i} {W}_{i}}$$

Formally, an additional similarity, Sc, can be calculated, as follows:

$$Ic = \frac{{\sum }_{i} ( {W}_{i} * {SIc}_{i} )}{{\sum }_{i} {W}_{i}} ,$$

where SIcᵢ is a binary quantity which equals 1 if pair i exists in the subject's data and 0 otherwise.

In some embodiments of the present invention the quantity SI comprises a correlation value between recorded activities. In some embodiments, the correlation value describes correlation between the activities recorded for the specific subject at the two locations associated with the pair, and in some embodiments the correlation value describes correlation between the activities recorded for the specific subject at any of the locations associated with the pair and the group activities as recorded at the same location. In some embodiments, the correlation value describes causality relations between activities.

Procedures for calculating correlation values, such as causality relations, are known in the art. In some embodiments of the present invention the Granger theory is employed [Granger C W J, 1969, “Investigating Causal Relations By Econometric Models And Cross-Spectral Methods,” Econometrica, 37(3):242]. Other techniques suitable for the present embodiments are found in Durka et al., 2001, “Time-frequency microstructure of event-related electroencephalogram desynchronisation and synchronisation,” Medical & Biological Engineering & Computing, 39:315; Smith Bassett et al., 2006, “Small-World Brain Networks” Neuroscientist, 12:512; He et al., 2007, “Small-World Anatomical Networks in the Human Brain Revealed by Cortical Thickness from MRI,” Cerebral Cortex 17:2407; and De Vico Fallani et al., “Extracting Information from Cortical Connectivity Patterns Estimated from High Resolution EEG Recordings: A Theoretical Graph Approach,” Brain Topogr 19:125; the contents of all of which are hereby incorporated by reference.

The connectivity weights assigned over the BNA pattern can be calculated as a continuous variable (e.g., using a function having a continuous range), or as a discrete variable (e.g., using a function having a discrete range or using a lookup table). In any case, connectivity weights can have more than two possible values. Thus, according to various exemplary embodiments of the present invention the weighted BNA pattern has at least three, or at least four, or at least five, or at least six edges, each of which being assigned with a different connectivity weight.

In some embodiments of the present invention the method proceeds to 16 at which a feature selection procedure is applied to the BNA pattern to provide at least one sub-set of BNA pattern nodes.

Feature selection is a process by which the dimensionality of the data is reduced by selecting the best features of the input variables from a large set of candidates that are most relevant for the learning process of an algorithm. By removing irrelevant data the accuracy of representing the original features of a data set is increased thereby enhancing the accuracy of data mining tasks such as predictive modeling. Existing feature selection methods fall into two broad categories known as forward selection and backward selection. Backward selection (e.g., Marill et al., IEEE Tran Inf Theory 1963, 9:11-17; Pudil et al., Proceedings of the 12th International Conference on Pattern Recognition (1994). 279-283; and Pudil et al., Pattern Recognit Lett (1994) 15:1119-1125) starts with all the variables and removes them one by one in a step-wise fashion to be left with the top-ranked variables. Forward selection (e.g. Whitney et al., IEEE Trans Comput 197; 20:1100-1103; Benjamini et al., Gavrilov Ann Appl Stat 2009; 3:179-198) starts with an empty variable set and adds the best variable at each step until any further addition does not improve the model.

In some embodiments of the present invention a forward selection of features is employed and in some embodiments of the present invention a backward selection features is employed. In some embodiments of the present invention the method employs a procedure for controlling the fraction of false positives that may lead to poor selection, such procedure is known as false discovery rate (FDR) procedure, and is found, for example, in Benjamini et al. supra, the contents of which are hereby incorporated by reference.

A representative example of a feature selection procedure suitable for the present embodiments is as follows. Initially, a group of subjects is considered (for example, either healthy controls or diseased subjects), optionally and preferably using a sufficiently large dataset to as to provide relatively high accuracy in representing the group. The group can be represented using a BNA pattern. The feature selection procedure is then applied on a training set of the dataset in order to evaluate each feature characterizing the group's dataset, wherein the evaluated feature can be a node of the BNA pattern or a pair of nodes of the BNA pair pattern or any combinations of nodes of the BNA pattern. The input to the feature selection algorithm is preferably evaluation scores (e.g., the score for each participant in the training set on each of the features) calculated using the training set. Feature selection can also be applied, on other features, such as, but not limited to, EEG and ERP features such as, but not limited to, coherence, correlation, timing and amplitude measures. Feature selection can also be applied on different combinations of these features.

The outcome of this procedure can be a set of supervised BNA patterns, each suitable to describe a different sub-group of the population with a specific set of features. The supervised BNA patterns obtained during the procedure can allow a comparison of the BNA pattern obtained for a single subject to a specific network or networks. Thus, the supervised BNA patterns can serve as biomarkers.

Once the BNA pattern is constructed it can be transmitted to a display device such as a computer monitor, or a printer. Alternatively or additionally, the BNA pattern can be transmitted to a computer-readable medium.

The method ends at 15.

### Annex 2

### Spatiotemporal Analysis by Parceling

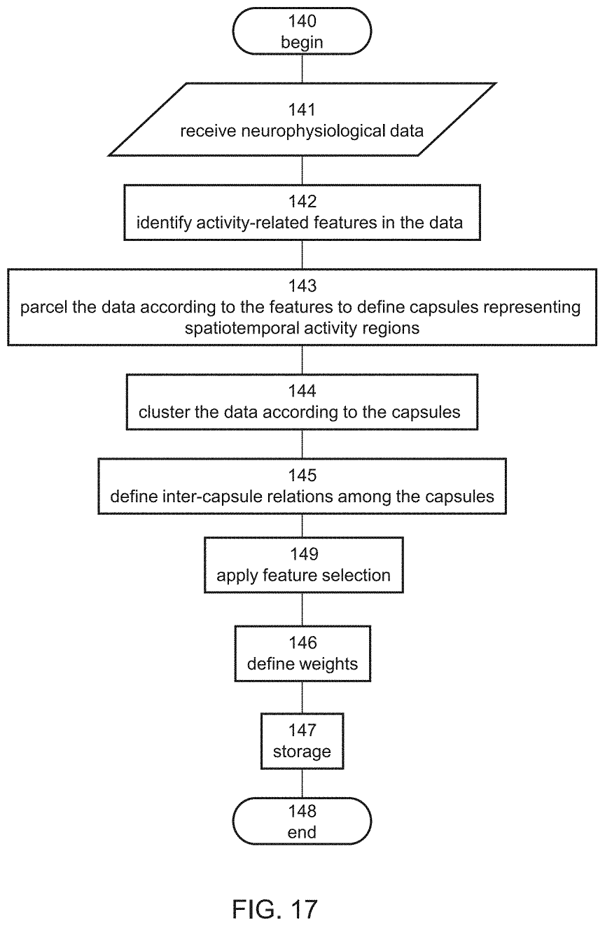

FIG. 17 is a flowchart diagram illustrating a method suitable for constructing a database from neurophysiological data recorded from a group of subjects, according to some embodiments of the present invention.

The neurophysiological data to be analyzed can be any data acquired directly from the brain of the subject under investigation, as further detailed hereinabove. The data can be analyzed immediately after acquisition (“online analysis”), or it can be recorded and stored and thereafter analyzed (“offline analysis”). The neurophysiological data can include any of the data types described above. In some embodiments of the present invention the data are EEG data. The neurophysiological data can be collected before and/or after the subject has performed or conceptualized a task and/or action, as further detailed hereinabove. The neurophysiological data can be used as event related measures, such as ERPs as further detailed hereinabove.

The method begins at 140 and optionally and preferably continues to 141 at which the neurophysiological data are received. The data can be recorded directly from the subject or it can be received from an external source, such as a computer readable memory medium on which the data are stored.

The method continues to 142 at which relations between features of the data are determined so as to identify activity-related features. The activity-related features can be extrema (peaks, through, etc.) and they can be identified as further detailed hereinabove.

The method continues to 143 at which a parcellation procedure is employed according to the identified activity-related features so as to define a plurality of capsules, each representing at least a spatiotemporal activity region in the brain. Broadly speaking, parcellation procedure defines a neighborhood of each identified feature. The neighborhood is optionally and preferably a spatiotemporal neighborhood. In some embodiments of the present invention the neighborhood is a spectral-spatiotemporal neighborhood, these embodiments are detailed hereinafter.

The neighborhood can be defined as a spatial region (two- or three-dimensional) in which the extremum is located and/or a time-interval during which the extremum occurs. Preferably, both a spatial region and time-interval are defined, so as to associate a spatiotemporal neighborhood for each extremum. The advantage of defining such neighborhoods is that they provide information regarding the spreading structure of the data over time and/or space. The size of the neighborhood (in terms of the respective dimension) can be determined based on the property of the extremum. For example, in some embodiments, the size of the neighborhood equals the full width at a predetermined fraction of the maximum, e.g., full width at half maximum (FWHM) of the extremum. Other definitions of the neighborhood are not excluded from the scope of the present invention.

In various exemplary embodiments of the invention a spatial grid is built over a plurality of grid elements. The input to the spatial grid built is preferably the locations of the measuring devices (e.g., locations on the scalp, epicortical surface, cerebral cortex or deeper in the brain). In various exemplary embodiments of the invention a piecewise interpolation is employed so as to build a spatial grid having a resolution which is higher than the resolution characterizing the locations of the measuring devices. The piecewise interpolation preferably utilizes a smooth analytical function or a set of smooth analytical functions.

In some embodiments of the present invention the spatial grid is a two-dimensional spatial grid. For example, the spatial grid can describe the scalp, or an epicortical surface or an intracranial surface of the subject.

In some embodiments of the present invention the spatial grid is a three-dimensional spatial grid. For example, the spatial grid can describe an intracranial volume of the subject.

Once the spatial grid is built, each identified activity-related feature is preferably associated with a grid element x (x can be surface element or a point location in embodiments in which a 2D grid is built, or a volume element or a point location in embodiments in which a 3D grid is built) and a time point t. A capsule corresponding to the identified activity-related feature can then be defined as a spatiotemporal activity region encapsulating grid elements nearby the associated grid element x and time points nearby the associated time point t. In these embodiments, the dimensionality of a particular capsule is D+1, where D is the spatial dimensionality.

The nearby grid elements optionally and preferably comprise all the grid elements at which an amplitude level of the respective activity-related feature is within a predetermined threshold range (for example, above half of the amplitude at the peak). The nearby time points optionally and preferably comprise all time points at which the amplitude level of the activity-related feature is within a predetermined threshold range, which can be the same threshold range used for defining the nearby grid elements.

The parceling 143 can optionally and preferably includes applying frequency decomposition to the data to provide a plurality of frequency bands, including, without limitation, delta band, theta band, alpha band, low beta band, beta band, and high beta band, as further detailed hereinabove. Higher frequency bands, such as, but not limited to, gamma band are also contemplated. In these embodiments, the capsules can be defined separately for each frequency band.

The present inventors also contemplate a parceling procedure in which each identified activity-related feature is associated with a frequency value f, wherein the capsule corresponding to an identified activity-related feature is defined as spectral-spatiotemporal activity region encapsulating grid elements nearby x, time points nearby t, and frequency values nearby f. Thus, in these embodiments, the dimensionality of a particular capsule is D+2, where D is the spatial dimensionality.

The definition of capsules according to some embodiments of the present invention is executed separately for each subject. In these embodiments, the data used for defining the capsules for a particular subject includes only the data collected from that particular subject, irrespective of data collected from other subjects in the group.

In various exemplary embodiments of the invention the method continues to 144 at which the data are clustered according to the capsules, to provide a set of capsule clusters. When the capsules are defined separately for each frequency band, the clustering is preferably also executed separately for each frequency band. The input for the clustering procedure can include some or all the capsules of all subjects in the group. A set of constraints is preferably defined, either a priori or dynamically during the execution of the clustering procedure, which set of constraints is selected to provide a set of clusters each representing a brain activity event which is common to all members of the cluster. For example, the set of constraints can include a maximal allowed events (e.g., one or two or three) per subject in a cluster. The set of constraints can also include a maximal allowed temporal window and maximal allowed spatial distance in a cluster. A representative example of a clustering procedure suitable for the present embodiments is provided in the Examples section that follows.

Once the clusters are defined, they can optionally and preferably be processed to provide a reduced representation of the clusters. For example, in some embodiments of the present invention a capsular representation of the clusters is employed. In these embodiments, each cluster is represented as a single capsule whose characteristics approximate the characteristics of the capsules that are the members of that cluster.

In some embodiments, the method proceeds to 145 at which inter-capsule relations among capsules are determined. This can be done using the procedure described above with respect to the determination of the edges of the BNA pattern (see, for example, FIGS. 16B-E). Specifically, the inter-capsule relations can represent causal relation between two capsules. For example, for each of a given pair of capsules, a time window can be defined. These time windows correspond to the width of the capsule along the time axis. A latency difference window between the two capsules can also be defined. This latency difference window corresponds to the separation along the time axis between the capsules.

The individual time windows and latency difference window can be used to define the relation between the pair of capsules. For example, a threshold procedure can be applied to each of these windows, so as to accept, reject or quantify (e.g., assign weight to) a relation between the capsules. The threshold procedure can be the same for all windows, or, more preferably, it can be specific to each type of window. For example, one threshold procedure can be employed to the width of the capsule along the time axis, and another threshold procedure can be employed to the latency difference window. The parameters of the thresholding are optionally dependent on the spatial distance between the capsules, wherein for shorter distance lower time thresholds are employed.

The present embodiments contemplate many types of inter-capsule relations, including, without limitation, spatial proximity between two defined capsules, temporal proximity between two defined capsules, spectral (e.g., frequency of signal) proximity between two defined capsules, and energetic (e.g., power or amplitude of signal) proximity between two defined capsules.

In some embodiments, a group capsule is defined for a group of subjects each having capsule and spatiotemporal peak. The relation between two group capsules is optionally and preferably defined based on the time difference between the respective group capsules. This time difference is preferably calculated between the corresponding two spatiotemporal peaks of subjects from both group capsules. This time difference can alternatively be calculated between the onsets of the spatiotemporal event activations of each of the capsules (rather than the time differences between peaks).

For example, the two group capsules can be declared as a pair of related capsules if the time difference between the capsules among subjects having those capsules is within a predefined time window. This criterion is referred to as the time-window constraint. A typical time-window suitable for the present embodiments is several milliseconds.

In some embodiments, the relation between two group capsules is defined based on the number of subjects having time those capsules. For example, the two group capsules can be declared as a pair of related capsules if the number of subjects having the capsules is above a predetermined threshold. This criterion is referred to as the subject number constraint. In various exemplary embodiments of the invention the both time window constraint and the subject number constraint are used in addition, wherein two group capsules are declared as a pair of related capsules when both the time window constraint and the subject number constraint are fulfilled. The maximum number of subjects that can create a particular pair of capsules is referred to as the intersection of subjects of the two groups.

Thus, in the present embodiments a capsule network pattern is constructed, which capsule network pattern can be represented as a graph having nodes corresponding to capsules and edges corresponding to inter-capsule relations.

In some embodiments of the present invention the method applies (operation 149) a feature selection procedure to the capsules to provide at least one sub-set of capsules.

In some embodiments of the present invention a forward selection of features is employed and in some embodiments of the present invention a backward selection features is employed. In some embodiments of the present invention the method employs a procedure for controlling the fraction of false positives that may lead to poor selection, such procedure is known as false discovery rate (FDR) procedure, and is found, for example, in Benjamini et al. supra, the contents of which are hereby incorporated by reference.

A representative example of a feature selection procedure suitable for the present embodiments is as follows. Initially, a group of subjects is considered (for example, either healthy controls or diseased subjects), optionally and preferably using a sufficiently large dataset to as to provide relatively high accuracy in representing the group. The group can be represented using a set of capsules. The feature selection procedure is then applied on a training set of the dataset in order to evaluate each feature or various combinations of features characterizing the group's dataset. The input to the feature selection algorithm is preferably evaluation scores (e.g., the score for each participant in the training set on each of the features) calculated using the training set. Feature selection can also be applied, on other features, such as, but not limited to, BNA pattern event-pairs, and EEG and ERP features such as, but not limited to, coherence, correlation, timing and amplitude measures. Feature selection can also be applied on different combinations of these features.

The outcome of this procedure can be a set of supervised network of capsules, each suitable to describe a different sub-group of the population with a specific set of features. The networks obtained during the procedure can allow a comparison of the capsules obtained for a single subject to a specific network or networks. Thus, the obtained networks obtained can serve as biomarkers.

In some embodiments of the invention, the method continues to 146 at which weights are defined for each cluster (or capsular representation thereof) and/or each pair of clusters (or capsular representations thereof). Weights for pairs of clusters can be calculated as described above with respect to the weights assigned to the edges of the BNA.

Weights for individual capsules or clusters can describe the existence level of the particular capsule in the database. For example, the weight of a cluster can be defined as the mean amplitude as calculated over all the capsules in the cluster. The weight is optionally and preferably normalized by the sum of all amplitude means of all clusters.

Also contemplated is a weight that describes the statistical distribution or density of one or more of the parameters that define the capsules in the cluster. Specifically, the weight can include at least one of: the distribution or density of the amplitudes over the cluster, the spatial distribution or spatial density over the cluster, the temporal distribution or temporal density over the cluster, and the spectral distribution or spectral density over the cluster.

At 147 the method stores the clusters and/or representations and/or capsule network pattern in a computer readable medium. When weights are calculated, they are also stored.

The method ends at 148.

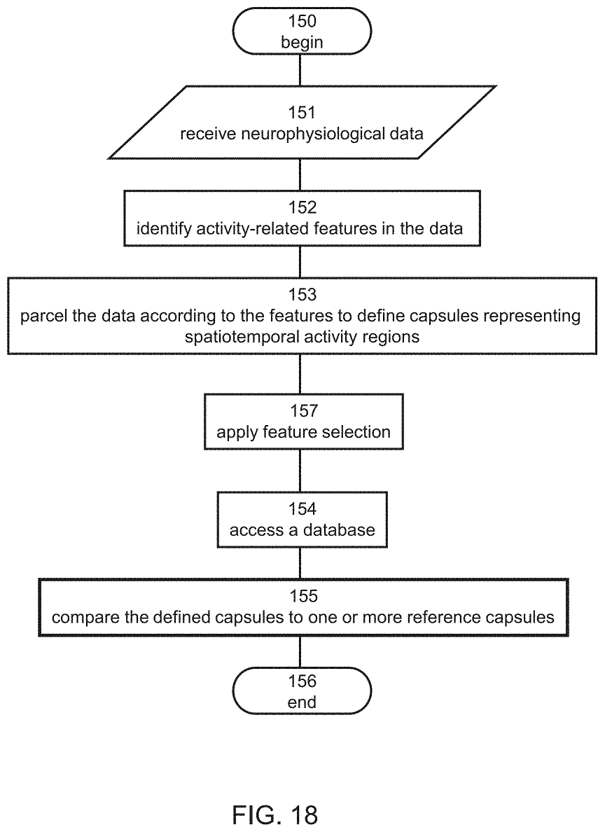

FIG. 18 is a flowchart diagram illustrating a method suitable for analyzing neurophysiological data recorded from a subject, according to some embodiments of the present invention.

The neurophysiological data to be analyzed can be any data acquired directly from the brain of the subject under investigation, as further detailed hereinabove. The data can be analyzed immediately after acquisition (“online analysis”), or it can be recorded and stored and thereafter analyzed (“offline analysis”). The neurophysiological data can include any of the data types described above. In some embodiments of the present invention the data are EEG data.

The neurophysiological data can be collected before and/or after the subject has performed or conceptualized a task and/or action, as further detailed hereinabove. The neurophysiological data can be used as event related measures, such as ERPs, as further detailed hereinabove.

The method begins at 150 and optionally and preferably continues to 151 at which the neurophysiological data are received. The data can be recorded directly from the subject or it can be received from an external source, such as a computer readable memory medium on which the data are stored.

The method continues to 152 at which relations between features of the data are determined so as to indentify activity-related features. The activity-related features can be extrema (peaks, through, etc.) and they can be identified as further detailed hereinabove.

The method continues to 153 at which a parcellation procedure is employed according to the identified activity-related features so as to define a plurality of capsules, as further detailed hereinabove. The capsules and the relations between capsules define a capsule network pattern of the subject, as further detailed hereinabove.

In some embodiments, the method proceeds to 157 at which a feature selection procedure is employed as further detailed hereinabove.

The method optionally and preferably continues to 154 at which a database having a plurality of entries, each having an annotated database capsule is accessed. The database can be constructed as described above with respect to FIG. 17.

The term “annotated capsule” refers to a capsule which is associated with annotation information. The annotation information can be stored separately from the capsule (e.g., in a separate file on a computer readable medium). The annotation information can be associated with a single capsule or a collection of capsules. Thus, for example, the annotation information can pertain to the presence, absence or level of the specific disorder or condition or brain function. Also contemplated are embodiments in which the annotation information pertains to a specific brain related disorder or condition in relation to a treatment applied to the subject. For example, a capsule (or collection of capsules) can be annotated as corresponding to a treated brain related disorder. Such capsule (or collection of capsules) can also be annotated with the characteristics of the treatment, including dosage, duration, and elapsed time following the treatment. A capsule (or collection of capsules) can optionally and preferably be annotated as corresponding to an untreated brain related disorder. Any of the disorders, conditions brain functions, and treatments described above can be included in the annotation information.

Alternatively or additionally, the capsule (or collection of capsules) can be identified as corresponding to a specific group of individuals (e.g., a specific gender, ethnic origin, age group, etc.), wherein the annotation information pertains to the characteristics of this group of individuals.

The database can include capsules defined using data acquired from a group of subjects, or it can capsules defined using data acquired from the same subject at a different time, for example, an earlier time. In the latter case, the annotation of the capsules can include the acquisition date instead or in addition to the aforementioned types of annotations.

The method proceeds to 155 at which at least some (e.g., all) of the defined capsules are compared to one or more reference capsules.

The present embodiments contemplate more than one type of reference capsules.

In some embodiments of the present invention the reference capsules are baseline capsules defined using neurophysiological data acquired from the same subject at a different time, for example, an earlier time.

A particular and non limiting example for these embodiments is the case of several treatment sessions, e.g., N sessions, for the same subject. Data can be acquired before and after each session and capsules can be defined for each data acquisition. The capsules defined before treatment can be used as baseline capsules to which capsules acquired from post treatment acquisition can be compared. In some embodiments of the present invention the baseline capsules are capsules defined from acquisition before the first session, wherein capsules defined from each successive acquisition are compared to the same baseline capsules. This embodiment is useful for assessing the effect of the treatment over time. In some embodiments of the present invention the baseline capsules are capsules defined from acquisition before the kth session, wherein capsules defined from an acquisition following the kth session are compared to these baseline capsules. This embodiment is useful for assessing the effect of one or more particular sessions.

In some embodiments of the present invention the reference capsules are capsules defined using neurophysiological data acquired form a different subject.

The variation of a particular capsule as defined from the data relative to the baseline capsule (for example, as defined previously, or as defined from previously acquired data), can be compared according to some embodiments of the present invention to variations among two or more capsules annotated as normal. For example, the variation of a particular capsule relative to the baseline capsule can be compared to a variation of a first capsule annotated as normal and a second capsule also annotated as normal. These annotated capsules are optionally and preferably defined from neurophysiological data acquired from different subjects identified as having normal brain function.

The advantage of these embodiments is that they allow assessing the diagnostic extent of the observed variations of a particular capsule relative to a baseline capsule. For example, when the variation relative to the baseline capsule are similar to the variations obtained from neurophysiological data among two or more different subjects identified as having normal brain functions, the method can assess that the observed variation relative to the baseline capsule are of reduced or no significance. On the other hand, when the variation relative to the baseline capsule are substantive compared to the variations among normal subjects, the method can assess that the observed variation relative to the baseline capsule are diagnostically significant.

In embodiments in which a database of previously annotated capsules is accessed (operation 154) the reference capsules are optionally and preferably the capsules of the database. The capsules can be compared to at least one database capsule annotated as abnormal, and at least one database capsule annotated as normal. A database capsule annotated as abnormal is a capsule which is associated with annotation information pertaining to the presence, absence or level of a brain related disorder or condition. A database capsule annotated as normal is a capsule which was defined using data acquired from a subject or a group of subjects identified as having normal brain function. Comparison to a database capsule annotated as abnormal and a database capsule annotated as normal is useful for classifying the subject according to the respective brain related disorder or condition. Such classification is optionally and preferably provided by means of likelihood values expressed using similarities between the respective capsules.

The comparison between capsules is typically for the purpose of determining similarity between the compared capsules. The similarity can be based on correlation between the capsules along any number of dimensions. In experiments performed by the present inventors, correlation between two capsules that were not even in their size was employed. These experiments are described in more detail in the Examples section that follows.

The comparison between capsules can comprise calculating a score describing the degree of similarity between the defined capsule and the capsules of the data base. When the database corresponds to a group of subjects having a common disorder, condition, brain function, treatment, or other characteristic (gender, ethnic origin, age group, etc.), the degree of similarity can express, for example, the membership level of the subject in this group. In other words, the degree of similarity expresses how close or how far are the disorder, condition, brain function, treatment, or other characteristic of the subject from that of the group.

The score calculation can include calculating of a statistical score (e.g., z-score) of a spatiotemporal vector corresponding to the subject's capsule using multidimensional statistical distribution (e.g., multidimensional normal distribution) describing the respective database capsule. In some embodiments of the present invention, the statistical score is weighed using the weights in the database. The score calculation can also include calculation of a correlation between capsule and a respective database capsule. A representative example of a scoring procedure suitable for the present embodiments is provided in the Examples section that follows.

The score of a particular score relative to the database can also be used for comparing two capsules two each other. For example, consider a first capsule C1 and a second capsule C2 which, a priori, is not the same as C1. Suppose that C1 is compared to database X and is assigned with a score S1. Suppose further that C2 is compared to a database Y (which, in some embodiments is database X, but may also be a different database) and is assigned with a score S2. The comparison between C1 and C2 is achieved according to some embodiments of the present invention by comparing S1 to S2. These embodiments are particularly useful when one of C1 and C2 is a baseline capsule, and when C1 and C2 are defined from neurophysiological data collected from different subjects.

The comparison between the subject's capsule and database capsules can be executed irrespective of any inter-capsule relation of any type. In these embodiments the subject's capsule is compared to the database capsules without taking into account whether a particular pair of database capsules has a relation in terms of time, space, frequency or amplitude.

Alternatively, the method can determine inter-capsule relations among the defined capsules, and construct a capsule network pattern responsively to the inter-capsule relations, as further detailed hereinabove. In these embodiments, the comparison is between the constructed pattern and the database pattern.

The comparison between the subject's capsule and database capsules is optionally and preferably with respect to the supervised network of capsules obtained during the feature selection procedure.

The method ends at 156.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims as well as Annexes 1-3.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of analyzing performance of an invasive brain stimulation tool having a plurality of electrode contacts, the method comprising:
obtaining encephalography data collected from a brain of a subject electrically stimulated by at least one of the electrode contacts;
segmenting the data into a plurality of epochs, each corresponding to a single stimulation event generated brain stimulation tool; and
applying a spatiotemporal analysis to said epochs so as to determine at least one of (i) location of said at least one electrode contact in said brain, and (ii) therapeutic effect of said at least one electrode contact.

2. The method of claim 1, wherein said encephalography data comprise EEG data.

3. The method of claim 1, wherein said encephalography data comprise MEG data.

4. The method according to claim 1, wherein said electrode contacts are electrode contacts of at least one DBS electrode.

5. (canceled)

6. The method according to claim 1, wherein at least one single stimulation event is generated by a single pulse applied by a single electrode contact.

7. (canceled)

8. The method according to claim 1, wherein at least one single stimulation event is generated by more than one electrode contacts, each applying a single pulse.

9. (canceled)

10. The method according to claim 1, wherein said segmenting comprises extracting stimulation pulses onsets from the data based on at least one of shapes and patterns of artifacts in the data.

11. (canceled)

12. The method according to claim 1, wherein said brain of said subject is stimulated at a frequency of at most 20 Hz, and wherein each epoch has a duration of at least 50 ms.

13. (canceled)

14. The method according to claim 1, wherein said brain of said subject is stimulated by one electrode contact at a time.

15. (canceled)

16. The method according to claim 1, wherein said brain of said subject is stimulated by two electrode contacts at a time, simultaneously.

17. (canceled)

18. The method according to claim 1, wherein said brain of the subject is stimulated by three electrode contacts at a time, simultaneously.

19. (canceled)

20. The method according to claim 1, wherein each stimulation event is characterized by a set of parameters, and wherein all stimulation events are characterized by the same set of values for said parameters.

21. (canceled)

22. The method according to claim 20, comprising repeating said obtaining, said segmenting, and said spatiotemporal analysis for a different set of values for said parameters.

23. (canceled)

24. The method according to claim 20, wherein said parameters comprise at least one of: stimulation intensity, stimulation frequency and stimulation directionality.

25. (canceled)

26. The method according to claim 1, wherein said spatiotemporal analysis comprises:
identifying activity-related features in said epochs;
parceling the data according to said activity-related features to define a plurality of capsules, each representing a spatiotemporal activity region in the brain; and
comparing capsules corresponding to different electrode contacts;
wherein said determination of said location and/or therapeutic effect is based, at least in part, on said comparison.

27. (canceled)

28. The method according to claim 26, wherein said comparison comprises calculating a similarity score among pairs of capsules.

29. (canceled)

30. The method according to claim 26, further comprising clustering said capsules to provide at least one cluster of capsules, wherein said determination of said location and/or therapeutic effect is based, at least in part, on a size of said at least one cluster.

31. (canceled)

32. The method according to claim 1, further comprising configuring a neurostimulator of said brain stimulation tool, based on said location and/or therapeutic effect.

33. (canceled)

34. The method according to claim 1, further comprising applying a time-frequency analysis to said epochs to provide time-frequency patterns, wherein said determination of said location is based on said time-frequency patterns.

35-37. (canceled)

38. The method according to claim 1, further comprising determining, based on said spatiotemporal and/or time-frequency analysis, at least one physiological event selected from the group consisting of increased tremor, and increased twitching.

39. (canceled)

40. A method of analyzing performance of a brain stimulation tool having a plurality of electrode contacts, the method comprising:
obtaining encephalography data collected from a brain of a subject electrically stimulated by at least one of the electrode contacts;
segmenting the data into a plurality of epochs, each corresponding to a stimulation event generated by a train of pulses transmitted by single electrode contact; and
calculating power spectral density for averages of said epochs so as to determine location of said at least one electrode contact in said brain.

41. The method according to claim 40, wherein said brain of said subject is stimulated intermittently at a frequency of at least 80 Hz.

42. The method according to claim 40, further comprising determining distribution of said encephalography data over a scalp of said subject, separately for at least one encephalographic frequency band, wherein said determination of said location is also based on said distribution.

43. (canceled)

44. A system for analyzing a brain stimulation tool having a plurality of electrode contacts, the system comprises a data processor configured for receiving encephalogram (EG) data recorded from a brain of a subject electrically stimulated by at least one of the electrode contacts, and executing the method according to claim 1.

45. A computer software product, comprising a computer-readable medium in which program instructions are stored, which instructions, when read by a data processor, cause the data processor to receive encephalogram (EG) data recorded from a brain of a subject electrically stimulated by at least one of the electrode contacts, and to execute the method according to claim 1.
